(19) United States
(12) Patent Application Publication
(10) Pub. No.: US 2003/0100068 A1
(43) Pub. Date: May 29, 2003

(54) RANKL mimics and uses thereof

(76) Inventors: Jonathan Lam, West Memphis, AR (US); F. Patrick Ross, Overland, MO (US); Steven L. Teitelbaum, St. Louis, MO (US)

Correspondence Address: SONNENSCHEIN NATH & ROSENTHAL, P.O. BOX 061080, WACKER DRIVE STATION, CHICAGO, IL, 60606-1080, US

(21) Appl. No.: 10/272,411

(22) Filed: Oct. 15, 2002

Related U.S. Application Data
(60) Provisional application No. 60/329,393, filed on Oct. 15, 2001.
(60) Provisional application No. 60/329,231, filed on Oct. 12, 2001.
(60) Provisional application No. 60/329,360, filed on Oct. 15, 2001.
(60) Provisional application No. 60/328,876, filed on Oct. 12, 2001.

(51) Int. Cl. C12P 21/02; C12N 5/06; C07K 14/715; C07H 21/04

(52) U.S. Cl. 435/69.1; 435/320.1; 435/325; 530/350; 536/23.5

(57) ABSTRACT

The present invention provides non-naturally occurring proteins that bind to RANK and polynucleotides encoding the same. The proteins of the invention can be used for enhancing bone formation by either inhibiting bone resorption or inducing osteogenesis.

This application is related to and claims the benefit of the following U.S. applications, which are incorporated herein by reference as if restated here in full: Ser. No. 60/277,855 filed Mar. 22, 2001; Ser. No. 10/105,057 filed Mar. 22, 2002; Ser. No. 60/311,163 filed Aug. 9, 2001; Ser. No. 10/215,446 filed Aug. 9, 2002; Ser. No. 60/329,231 filed Oct. 12, 2001; Ser. No. 60/329,393 filed Oct. 15, 2001; Ser. No. 60/329,360 filed Oct. 15, 2001; Ser. No. 60/328,876 filed Oct. 12, 2001; U.S. non-provisional application entitled Methods for Screening Osteogenic Compounds, Lam, et al., filed Oct. 15, 2002; and U.S. non-provisional application entitled Bone Anti-Resorptive Compounds, Lam, et al., filed Oct. 15, 2002.

This invention was made in part with U.S. Government support under National Institutes of Health Grants AR32788, AR46123 and DE05413. The Government has certain rights in the invention.

### FIELD OF INVENTION

The present invention relates to recombinant polynucleotides, proteins encoded by such polynucleotides, and methods for producing the proteins that bind to the cell surface receptor RANK that is found on osteoblasts, osteoclasts and their precursors. The proteins of the invention can be used in methods for enhancing processes of bone formation or inhibiting bone resorption, thereby providing novel treatments for diseases or conditions which are at least partially characterized by loss of bone mass.

### BACKGROUND

Various conditions and diseases which manifest themselves in bone loss or thinning are a critical and growing health concern. It has been estimated that as many as 30 million Americans and 100 million worldwide are at risk for osteoporosis alone. Mundy, et al., Science, 286: 1946-1949 (1999). Other conditions known to involve bone loss include juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, corticosteroid treatment, metastatic bone diseases, periodontal bone loss, bone loss due to cancer, age-related loss of bone mass, and other forms of osteopenia. Additionally, new bone formation is needed in many situations, e.g., to facilitate bone repair or replacement for bone fractures, bone defects, plastic surgery, dental and other implantations and in other such contexts.

Bone is a dense, specialized form of connective tissue. Bone matrix is formed by osteoblast cells located at or near the surface of existing bone matrix. Bone is resorbed (eroded) by another cell type known as the osteoclast (a type of macrophage). These cells secrete acids, which dissolve bone minerals, and hydrolases, which digest its organic components. Thus, bone formation and remodeling is a dynamic process involving an ongoing interplay between the creation and erosion activities of osteoblasts and osteoclasts. Alberts, et al., Molecular Biology of the Cell, Garland Publishing, N.Y. (3rd ed. 1994), pp. 1182-1186.

Present forms of bone loss therapy are primarily anti-resorptive, in that they inhibit bone resorption processes, rather than enhance bone formation. Among the agents which have been used or suggested for treatment of osteoporosis because of their claimed ability to inhibit bone resorption are estrogen, selective estrogen receptor modulators (SERM's), calcium, calcitriol, calcitonin (Sambrook, P., et al., N. Engl. J. Med. 328:1747-1753), alendronate (Saag, K., et al., N. Engl. J. Med. 339:292-299) and other bisphosphonates. Luckman, et al., J. Bone Min. Res. 13, 581 (1998). However, anti-resorptives fail to correct the low bone formation rate frequently involved in net bone loss, and may have undesired effects relating to their impact on the inhibition of bone resorption/remodeling or other unwanted side effects.

As a result, it would be very desirable to obtain other compounds for treatment of bone loss. There is a need both for additional anti-resorptive compounds and compounds with osteogenic activity that might be used to develop therapeutics inhibiting bone loss or enhancing bone formation. Unfortunately, the number of assays currently available for screening and identifying potential osteogenic agents is very limited. One such assay is disclosed in U.S. Pat. No. 6,083,690, and it determines the osteogenic potential of a compound based on its ability to stimulate bone cells to produce bone growth factors in the bone morphogenetic family. Another is taught in U.S. non-provisional application entitled Methods for Screening Osteogenic Compounds, Lam, et al., filed Oct. 12, 2002.

A key development in the field of bone cell biology is the recent discovery that RANK ligand which is expressed on stromal cells, osteoblasts, activated T-lymphocytes and mammary epithelium, is the unique molecule essential for differentiation of macrophages into osteoclasts. Lacey, et al., Cell 93: 165-176 (1998)(Osteoprotegerin Ligand is a Cytokine that Regulates Osteoclast Differentiation and Activation.). RANKL has several functions and in the early literature is variously called osteoprotegerin ligand (OPGL), TNF-related activation induced cytokine (TRANCE), or osteoclast differentiation factor (ODF). The cell surface receptor for RANKL is RANK, Receptor Activator of Necrosis Factor (NF)-kappa B. RANKL is a type-2 transmembrane protein with an intracellular domain of less than about 50 amino acids, a transmembrane domain of about 21 amino acids, and an extracellular domain of about 240 to 250 amino acids. RANKL exists naturally in transmembrane and soluble forms. The deduced amino acid sequence for at least the murine, rat and human forms of RANKL-and variants thereof are known. See, e.g., Anderson, et al., U.S. Pat. No. 6,017,729, Boyle, U.S. Pat. No. 5,843,678, and Xu J., et al., J. Bone Min. Res. (2000/15:2178) each of which is incorporated herein by reference in its entirety. Furthermore, we have solved the crystal structure of RANKL ectodomain, as disclosed in provisional application Ser. No. 60/311,163, filed Aug. 9, 2001.

RANKL has been identified as a potent inducer of bone resorption and as a positive regulator of osteoclast development. Lacey, et al., supra. In addition to its role as a factor in osteoclast differentiation and activation, RANKL has been reported to induce human dendritic cell (DC) cluster formation. Anderson, et al., supra, and mammary epithelium development J. Fata, et al., “The osteoclast differentiation factor osteoprotegerin ligand is essential for mammary gland development,” Cell, 103:41-50 (2000). Recently, we have determined that specific forms of RANKL play a role in anabolic bone formation processes and can be utilized in methods for stimulation of osteoblast proliferation or bone nodule mineralization, as disclosed in provisional application Ser. No. 60/277,855, filed Mar. 22, 2001. In addition, the current patent application discloses methods for stimulation of osteogenesis using specific modified forms of RANKL or mimics thereof, thereby providing novel methods of treating, preventing or inhibiting bone loss in patients. Due to the paucity of anabolic bone agents, it would be desirable to discover or develop other compounds besides RANK ligand fusion proteins that can play a role in enhanced bone formation. In addition, the current patent application discloses methods for inhibiting osteoclast activity and decreasing bone loss.

Accordingly, a need exists for novel methods and compositions which are useful in treating diseases at least partially characterized by loss of bone mass.

### SUMMARY OF THE INVENTION

The present invention relates to non-naturally occurring proteins that contain various external surface loops of RANKL and that bind RANK (“RANKL mimics”), polynucleotides encoding RANKL mimics, and methods for producing RANKL mimics. One or more of the RANKL loops, in combination with a heterologous protein core obtained from a non-RANKL member of the TNF superfamily, provide mimics of RANKL. Native RANKL is a self-assembling homotrimer that upon binding RANK induces formation of a RANK triad. An “oligomeric RANKL mimic” is a RANKL mimic that can bind and cluster a multiplicity of RANK triads. A “monomeric RANKL mimic” is a RANKL mimic that while binding to RANK does not form RANK triads or multiples of triads. Oligomeric RANKL mimics can be used to induce osteogenesis by causing the development and activation of osteoblasts. Monomeric RANKL mimics can be used to compete with native RANKL to block the formation of RANK triads and thereby block osteoclast development. Polynucleotides of SEQ ID NO: 1 and SEQ ID NO: 51 both encode natural variants of human RANKL.

Accordingly, among the objects of the invention is the provision of recombinant DNA molecules that encode polypeptides that bind to RANK and have one or more of the external surface loops, AA″, EF, CD, and/or DE, of RANKL. Said recombinant DNA molecules may comprise DNA sequences encoding non-RANKL, TNF superfamily proteins including, without limitation, CD40L, TRAIL, Fas ligand, TNFα, TNFβ, Lymphotoxin, Lymphotoxin β, EDA-A1, EDA-A2, BLyS/BAFF/TALL-1, OX40L, CD27L, CD30L, 4-1 BB L, TWEAK, LIGHT, VEGI, AITRL, APRIL/TALL-2, TL1A and those represented by SEQ ID NO: 2-SEQ ID NO: 18. At least one or more portions of said sequence that encode external surface loops are substituted with one or more of the polynucleotide sequences encoding the external surface loops AA″, EF, CD, and/or DE of RANKL (SEQ ID NO: 1 or SEQ ID NO: 51). Preferably, the recombinant DNA sequences comprise DNA sequences of proteins of the TNF superfamily wherein one or more of the portions of said sequences which encode the external surface loops are substituted with one or more of the polynucleotide sequences encoding the external surface loops AA″, EF, CD, and/or DE of RANKL (SEQ ID NO: 1 or SEQ ID NO: 51).

Alternatively, some DNA subsequences encoding TNF superfamily proteins that encode surface loops may be excised without substitution, or may be substituted with DNA encoding unrelated polypeptide domains, while one or more others may be substituted with RANKL surface loop-encoding sequences of SEQ ID NO: 1 or SEQ ID NO: 51.

In a particularly preferred embodiment, some DNA subsequences encoding surface loops of TNF superfamily proteins may be substituted with polynucleotide sequences encoding other functional domains, such as an oligomerization domain, while one or more others are substituted with RANKL surface loop-encoding sequences of SEQ ID NO: 1 or SEQ ID NO: 51 in order to bind RANK. This is expected to form oligomers of RANK trimers and to trigger osteogenic activity as taught in U.S. application Ser. No. 60/277,855, filed Mar. 22, 2001 and incorporated herein by reference. Alternatively, other modifications of TNF superfamily nucleotides and proteins may be undertaken, such as addition of a 5′ polynucleotide encoding a GST or other moiety in addition to the above discussed loop substitutions, in order to encode polypeptides mimicking the compounds taught in Example 2 and in U.S. application Ser. No.60/277,855, filed Mar. 22, 2001. In particular, a preferred embodiment comprises replacement of AA″, EF, and CD loops of TALL-1/BAFF/BLYS with the AA″, EF, and/or CD loops of RANKL (SEQ ID NO: 1 or SEQ ID NO: 51) to target RANK, while leaving the oligomerizing DE loop of TALL-1 intact (see U.S. application Ser. No. 60/277,855, filed March 22, 2001, and references therein).

Additional preferred embodiments comprise polynucleotide sequences encoding RANKL mimics also having amino acid modifications at the interfaces mediating trimerization. This results in TNF superfamily monomers that bind RANK but fail to trimerize. These monomeric RANKL mimics may compete for binding with native trimeric RANKL, thereby inhibiting induction of intracellular signals by endogenous RANKL.

The polynucleotides comprise polynucleotides encoding a TNF superfamily cytokine, other than RANKL, wherein one or more of the portions of said polynucleotide which encode external surface loops are substituted with one or more of the polynucleotide sequences encoding the external surface loops AA″, EF, CD, and/or DE of RANKL (SEQ ID NO: 1 or SEQ ID NO: 51). This may be accomplished for any TNF superfamily cytokine in a manner similar to that illustrated for TNFα in Example 4. Polynucleotides according to the invention can be single or double stranded and, when single stranded, can be either the coding strand or the complementary non-coding strand. They may be deoxyribonucleic acids or ribonucleic acids.

Depending on the substitution or modification, proteins encoded by these polynucleotides can bind RANK and activate downstream signals or compete with the binding of native RANKL, thereby inhibiting downstream signals. Alternatively, as in Example 3, some substitutions may delay internalization and prolong activation, while others may conceivably decrease activation time. Since both osteoclasts and osteoblasts express RANK on their surfaces, such compounds might be envisioned to either inhibit bone resorption or to stimulate bone formation, or both. Prolonged activation can be used to promote osteogenic activity, while transient activation promotes osteoclastic activity.

The invention also encompasses expression vectors comprising the recombinant DNA molecules of the present invention, and host cells comprising such expression vectors. In a preferred embodiment, the expression vectors comprise polynucleotides of the TNF superfamily wherein one or more of the portions of which encoding external surface loops having been substituted with one or more of the polynucleotide sequences encoding the external surface loops AA″, EF, CD, and/or DE of SEQ ID NO: 1 or SEQ ID NO: 51. In another preferred aspect, the host cells comprise said expression vectors.

### BRIEF DESCRIPTION OF FIGURES

FIG. 1 is a structure-based alignment of TNF family cytokines, including TRAIL, CD40L, TNFα, TNFβ and ACRP30, with RANKL.

FIG. 2 is a depiction of a schematic of stereo ribbon diagrams of the RANKL monomer in comparison with those of TNF and TRAIL.

FIG. 3 is a graphic presentation of alkaline phosphatase (AP) activity following RANKL exposure.

FIG. 4 depicts GST-RANKL as oligomeric complexes, whereas cleaved RANKL (GST removed) does not exist in oligomeric forms, but solely as a mono-trimer.

FIG. 4(a) depicts chromatograph results showing that cleaved RANKL migrates as a single trimeric species (1n) while GST-RANKL exists as a poly disperse mixture of non-covalently associated mono-trimeric (1n) and oligomeric (2-100 n) units under dynamic equilibrium.

FIG. 4(b) depicts possible oligomeric structures of the GST-RANKL complex.

FIG. 5 consists of confocal microscopy images showing that cleaved RANKL/RANK complexes are rapidly internalized, whereas GST-RANKL/RANK complexes remain on the cell surface for at least one hour. On the merged images, colocalization of RANK (green fluorescence) and cell surface (red fluorescence) appears yellow.

### DETAILED DESCRIPTION

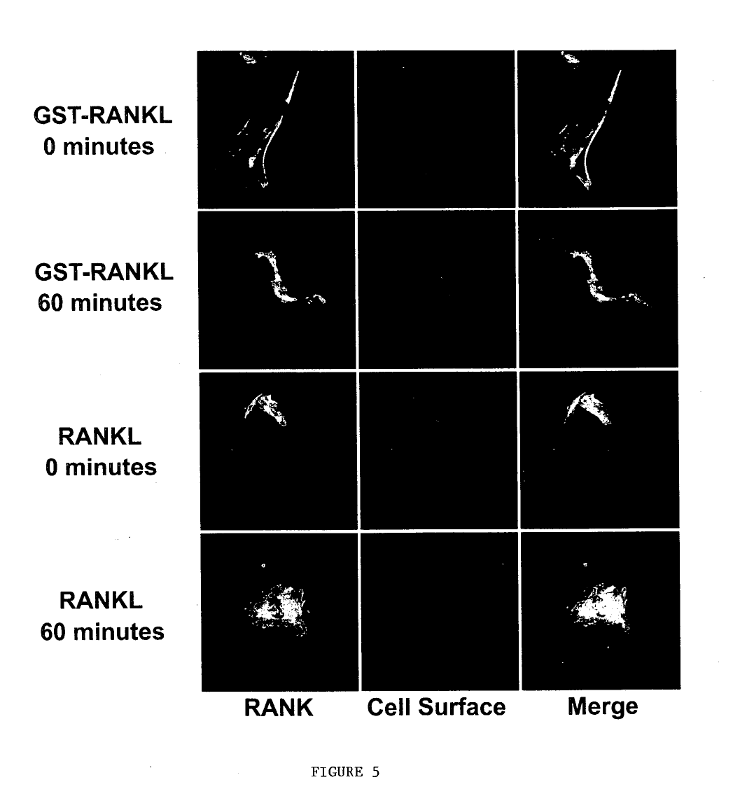

The present invention is based on applicants' discovery that the interaction between certain oligomerized RANKL fusions proteins and RANK on osteoblasts or osteoblast precursors results in accelerated rate of bone formation. Specifically, mice treated with a fusion product of the external domain of RANKL and glutathione S-transfersase (GST-RANKL) were shown to exhibit activation of osteoblasts and increased bone density. Applicants' further discovery of the identity and structure of the operative external loops of RANKL allows for the synthesis and use of RANKL mimics embodying that invention. Native RANKL is a self-assembling homotrimer that upon binding RANK induces formation of a RANK triad, leading to the activation of the downstream signaling pathway. While not being bound to a particular theory, oligomeric RANKL mimics induce clustering of multiple RANK triads, resulting in decreased internalization of the RANK receptor, as demonstrated in FIG. 5. This prolonged surface residency of RANK leads to prolonged activation on osteoblasts and their precursors, which stimulates bone formation. The invention also pertains to monomeric RANKL mimics, which, while binding RANK, do not form activated RANK triads. Monomeric RANKL mimics can be used to block the signaling of native RANKL and, thereby, to block osteoclast activity.

The interaction between native RANKL and RANK results in the activation of NFkB and ERK intracellular signal pathways, among others. The time course of intracellular protein activity, especially ERK activity, when osteoblasts are exposed to the oligomeric RANKL, GST-RANKL, is different from that observed in osteoclast precursors which also express RANK on the surface. In osteoclast precursors, ERK activity peaks 5-15 minutes after RANK/GST-RANKL interaction, and returns to basal levels after 15-30 minutes. In contrast, the ERK activity in osteoblasts peaks at 10 minutes after the same interaction, and is still above the basal level after 60 minutes. The prolongation of the time course is even more prominent in osteoblast precursor cells, wherein the demonstrated activity of ERK had not reached its maximum even 60 minutes after the RANK/oligomeric GST-RANKL interaction. Besides the different time course of ERK activity, osteoblasts and osteoblast precursor cells also exhibit prolonged activity of kinases such as IKK, PI3 kinase, Akt, p38 and JNK. This osteoblast-related activity contrasts with GST-RANKL interaction with RANK on osteoclasts, which results in short-lived activity of MAP kinases and bone resorption. While not being bound to a particular theory, it therefore appears that the prolonged activity of kinases observed in osteoblasts following GST-RANKL stimulation plays a role in the anabolic bone processes.

It is known that TNF family cytokine-induced intracellular signaling is attenuated by internalization of the receptor-ligand complex (see, e.g., Higuchi, M and Aggarwal, B. B., J. Immunol., 152:3550-3558 (1994)). Complexes comprising GST-RANKL are not internalized as promptly as complexes comprising RANKL, thus allowing for a longer interaction with the receptor and prolonged intracellular signaling as shown in FIG. 5 and Example 3. One embodiment of the present invention provides for RANKL mimics that may be utilized in such oligomerized complexes. In a particularly preferred embodiment, some DNA subsequences encoding surface loops of TNF superfamily members may be substituted with polynucleotide sequences encoding other functional domains, such as an oligomerization domain, while one or more others are substituted with RANKL surface loop-encoding sequences of SEQ ID NO: 1 or SEQ ID NO: 51 in order to bind RANK. These modifications are expected to enable formation of oligomers of RANKL trimers and to trigger osteogenic activity as taught in U.S. application Ser. No. 60/277,855, filed Mar. 22, 2001 and incorporated herein by reference in its entirety. Alternatively, other modifications of nucleotide sequences encoding surface loops of TNF superfamily members may be undertaken, such as addition of a 5′ polynucleotide encoding a GST or other moiety in addition to the above discussed loop substitutions, in order to encode polypeptides mimicking the compounds taught in Example 2 and in U.S. application Ser. No. 60/277,855, filed Mar. 22, 2001. In particular, a preferred embodiment comprises replacement-of AA″, EF, and CD loops of TALL-1/BAFF/BLYS as necessary to target RANK, while leaving the oligomerizing DE loop of TALL-1 intact (see U.S. application Ser. No. 60/277,855, filed Mar. 22, 2001, and references therein). Separate embodiments comprise replacement of the DE loop of any TNF superfamily member, including RANKL, with the oligomerizing DE loop of TALL-1, with the remaining external domains AA″, EF, and CD of RANKL. Oligomerization may be accomplished as described herein or by other appropriate means as known in the art.

In addition to oligomerization, other appropriate techniques for stabilizing and/or delaying internalization of the protein, such as tethering, may be used to like effect with the RANKL mimics of the present invention. Similarly, known treatments to increase stability or other beneficial characteristics of proteins, such as treatment with polyethylene glycol, or expression as an Fc fusion protein, may be utilized to like effect with the RANKL mimics of the present invention.

A recombinant polynucleotide can be constructed that encodes a RANKL mimic comprising a fusion protein based on the sequence of a member of the TNF superfamily in which the external loop sequences are replaced by one or more of the external surface loops AA″, EF, CD, and/or DE of RANKL. Said recombinant DNA molecules may comprise DNA sequences selected from the group encoding TNF superfamily members, including without limitation, CD40L, TRAIL, Fas ligand, TNFα, TNFβ, Lymphotoxin, Lymphotoxin β, EDA-A1, EDA-A2, BLyS/BAFF/TALL-1, OX40L, CD27L, CD30L, 4-1 BB L, TWEAK, LIGHT, VEGI, AITRL, APRIL/TALL-2, TL1A, those represented by SEQ ID NO: 2-SEQ ID NO: 18, and those yet to be discovered, wherein at least one or more portions of said sequence which encode external surface loops are substituted with one or more of the polynucleotide sequences encoding the external surface loops AA″, EF, CD, and/or DE of RANKL (SEQ ID NO: 1 or SEQ ID NO: 51). Preferably, the recombinant DNA sequences comprise DNA sequences of CD40L, TRAIL, TNFα, TNFβ, or ACRP30, wherein one or more of the portions of said sequences which encode the external surface loops are substituted with one or more of the polynucleotide sequences encoding the external surface loops AA″, EF, CD, and/or DE of SEQ ID NO: 1 or SEQ ID NO: 51. The “core” of a TNF superfamily member is the external domain of the member excluding the external loops. The external domain of human RANKL is between at least residues 162 and 313. From one to five additional amino acids on either the N- or C-terminal or both can be added without alteration of the properties.

Moreover, non-TNF super family proteins may be utilized to supply the core structure of the mimic. Such non-TNF proteins preferably assume substantially the core structure of the TNF super family. Preferably the non-TNF proteins will have a long serum half-life. Alternatively, any protein such as albumin may serve as well.

Alternatively, some DNA subsequences encoding surface loops of TNF superfamily members may be excised without substitution, or be substituted with DNA encoding unrelated polypeptide domains, while one or more others may be substituted with RANKL surface loop-encoding sequences of SEQ ID NO: 1 or SEQ ID NO: 51.

In a particularly preferred embodiment, DNA subsequences encoding surface loops of TNF superfamily proteins may be substituted with polynucleotide sequences encoding other functional domains, such as an oligomerization domain, while one or more others are substituted with RANKL surface loop-encoding sequences of SEQ ID NO: 1 or SEQ ID NO: 51 in order to bind RANK. This is expected to form oligomers of RANKL trimers and to trigger osteogenic activity as taught in U.S. application Ser. No. 60/277,855, filed Mar. 22, 2001 and incorporated herein by reference. Alternatively, other modifications of polynucleotides encoding TNF superfamily cytokines may be undertaken, such addition of a 5′ polynucleotide encoding a GST, leucine zipper or other moiety in addition to the above discussed loop substitutions, in order to encode polypeptides mimicking the compounds taught in U.S. application Ser. No. 60/277,855, filed Mar. 22, 2001. Oligomerization domains situated at the 3′ end are also conceived.

Depending on the substitution or modification, whether one discussed above or obvious, given these teachings, to one skilled in the art, proteins encoded by these polynucleotides may be envisioned to bind RANK and either to activate downstream signals or to compete with the binding of native RANKL, thereby inhibiting downstream signals. Since both osteoclasts and osteoblasts express RANK on their surface, such compounds might be envisioned to either inhibit bone resorption or to stimulate bone formation or both.

The invention also encompasses expression vectors comprising the recombinant DNA molecules of the present invention, and host cells comprising such expression vectors. In a preferred embodiment, the expression vectors comprise DNA sequences encoding TNF family proteins including but not limited to TRAIL, CD40L, TNFα, TNFβ, or ACRP30, wherein one or more of the polynucleotide subsegments encoding external surface loops having been substituted with one or more of the polynucleotide sequences encoding the external surface loops AA″, EF, CD, and/or DE of SEQ ID NO: 1 or SEQ ID NO: 51. In another preferred aspect, the host cells comprise said expression vectors.

The TNF superfamily ligands self-assemble into noncovalent trimers, such that each monomer of the ligand assumes a “jellyroll” β sandwich fold. The different members of the superfamily exhibit 25%-30% amino acid similarity, largely confined to inner surfaces involved in trimer assembly.

For the TNF superfamily members, RANK ligand, TRAIL, CD40L, ACRP30, specific residues have been identified in the solvent-nonaccessible inner surfaces which mediate formation of homotrimers. Lam, J., et al., Crystal Structure of the TRANCE/RANKL Cytokine Reveals Determinants of Receptor-Ligand Specificity. Journal of Clinical Investigation Vol. 108(7): 971-979 (2001); Cha S. S., et al., 2.8 Angstrom Resolution Crystal Structure of Human TRAIL, A Cytokine with Selective Anti-Tumor Activity, Immunity Vol. 11: 253-261 (1999); Karpusas, M., et al., 2 Angstrom Crystal Structure of an Extracellular Fragment of Human CD40 Ligand, Structure Vol. 3: 1031-1039 (1995); Shapiro, L. and Scherer, P. E., Crystal Structures of a Complement—1Q Family Protein Suggests an Evolutionary Link to Tumor Necrosis Factors, Current Biology Vol. 8: 335-338 (1998). These are believed to be representative of the TNF superfamily proteins. By similar methods, such domains in other TNF superfamily proteins may be identified.

Beta strands involved in the formation of inner surfaces exhibit significant topological homology, whereas the external surface loops of the trimeric ligands show little sequence or topological homology. As a result, substituting the external loops of any protein or mimetic which assumes the core structure of a TNF family ligand with the RANKL external loops while keeping the internal “jellyroll” structures (β strands) intact is believed to result in binding of said ligand to RANK. Monomeric RANKL mimics can be formed by substitutions in the conserved jellyroll portion of the core structure that affect monomer-monomer interactions. It is understood that other modifications, such as substitution of hydrophobic with hydrophilic amino acids, should preferably be included to inhibit any non-specific aggregation.

Thus, the present invention provides the polynucleotide sequence of TNF family ligands that can be modified in such manner as to express RANKL surface loops. It is to be noted that the invention includes but is not limited to polynucleotide sequences disclosed herein due to the fact that novel members of TNF superfamily of proteins may still be discovered. Polynucleotides encoding TNF superfamily members may be modified in such manner as to replace their external surface loops encoding for receptor specificity with external surface loops AA″, EF, CD, and DE of RANKL, the polynucleotide encoding sequences of which are shown in SEQ ID NO: 1 or SEQ ID NO: 51.

The external (solvent-accessible) surface loops of RANKL protein (SEQ ID NO: 19) are unique within the TNF family, displaying markedly divergent lengths and conformations: the AA″ loop (residues 170-193 of murine RANKL protein) bridges strands A and A″, the CD loop (residues 224-233) connects strands C and D, the EF loop (residues 261-269) links strands E and F, and the loop DE (residues 245-251) connects strands D and E.

Representatives examples of the surface loop polypeptide residue sequences of the four unique loops of RANKL and the analogous loops, replaceable under the present invention, from 5 other TNF superfamily members are as shown in Table 1:

TABLE 1

| | |
|---|---|
| RANKL (mouse) | |
| AA″-NAASIPSGSHKVTLSSWYHDRGWA | (SEQ ID NO 20) |
| CD-HETSGSVPTD | (SEQ ID NO 21) |
| DE-SIKIPSS | (SEQ ID NO 22) |
| EF-KNWSGNSEF | (SEQ ID NO 23) |
| TRAIL (human) | |
| AA″- | (SEQ ID NO 24) |
| TRGRSNTLSSPNSKNEKALGRKINSWESSRSGHS | |
| CD-QEEIKENTKN | (SEQ ID NO 25) |
| DE-TSYPDP | (SEQ ID NO 26) |
| EF-SCWSKDAEY | (SEQ ID NO 27) |
| CD40L (human) | |
| AA″-EASSKTTSVLQWAEKGYY | (SEQ ID NO 28) |
| CD-NREASS | (SEQ ID NO 29) |
| DE-SPGRFE | (SEQ ID NO 30) |
| EF-HSSAKPC | (SEQ ID NO 31) |
| TNF-alpha (mouse) | |
| AA″-NHQVEEQLEWLSQRANA | (SEQ ID NO 32) |
| CD-QGCPD | (SEQ ID NO 33) |
| DE-AISYQEK | (SEQ ID NO 34) |
| EF-PCPKDTPEGAELKP | (SEQ ID NO 35) |
| TNF-beta (human) | |
| AA″-DPSKQNSLLWRANTDRA | (SEQ ID NO 36) |
| CD-KAYSPKATSS | (SEQ ID NO 37) |
| DE-SSQYPFH | (SEQ ID NO 38) |
| EF-VYPGLQEP | (SEQ ID NO 39) |
| ACRP30 (human) | |
| AA″-ETRVTVPNVPIRFTKIF | (SEQ ID NO 40) |
| CD-none | |
| DE-D | (SEQ ID NO 41) |
| EF-YQEK | (SEQ ID NO 42) |
| RANKL (human) | |
| AA″-NATDIPSGSHKVSLSSWYHDRGWA | (SEQ ID NO 43) |
| CD-HETSGDLATE | (SEQ ID NO 44) |
| DE-SIKIPSS | (SEQ ID NO 45) |
| EF-KYWSGNSEF | (SEQ ID NO 46) |
| TNF-alpha (human) | |
| AA″-NPQAEGQLQWLNRRANA | (SEQ ID NO 47) |
| CD-QGCPS | (SEQ ID NO 48) |
| DE-AVSYQTK | (SEQ ID NO 49) |
| EF-PCQRETPEGAEAKP | (SEQ ID NO 50) |
| RANKL (human) | |
| AA″-NATDIPSGSHKVSLSSWYHDRGWG | (SEQ ID NO 52) |

It is recognized that cross-reactivity exists among TNF family ligands and receptors from different species. It is anticipated that surface loops from TNF superfamily ligands from species other than those explicitly listed are likely to be of similar utility. Identification of the exterior loops appropriate for replacement under the instant invention may be accomplished by structure-based alignment of TNF superfamily cytokines with RANKL by pairwise topological residue superimposition of the crystal structure of RANKL with those of the family member. This method was used to generate the analogous loop information contained in Table 1 for TRAIL (1d4v), CD40L (1aly), TNF-alpha (2tnf), TNF-beta (1tnr), and ACRP30 (1c28). The four character codes within parentheses are the PDB (Protein Data Dank) accession codes for the respective crystal structure atomic coordinates.

RANKL possesses a longer AA″ loop and a shorter EF loop than the typical TNF family member. The AA″ loop, together with the displacement of the CD loop confers a unique surface to the upper third of the RANKL molecule, whereas a subtle shift of the DE loop shapes the receptor binding groove at the base of RANKL molecule. For a detailed description of RANKL loops and binding specificity of RANK/RANKL interaction see U.S. provisional application Ser. No. 60/311,163, filed Aug. 9, 2001 and U.S. application Ser. No. 10/215,446 filed Aug. 9, 2002, incorporated by reference herein. Due to the homology between human and murine forms of RANKL, the identity of external surface loops of human RANKL can be easily determined. The methods described herein can be applied to external surface loops of any protein that assumes the core structure of a TNF family ligand, regardless of the organism from which the protein was isolated. However, in a preferred embodiment, the external surface loops of human RANKL molecule are used to substitute the external loops of any protein or mimetic which assumes the core structure of a TNF family ligand while keeping the internal core “jellyroll” structures (β strands) of such protein intact.

In certain circumstances, replacement of these external surface loops may be replaced, thereby generating a RANKL mimic that binds efficiently but is deficient in ability to activate the receptor. Such a RANKL mimic could be utilized to compete with endogenous RANKL and function to decrease RANKL pathway signaling in an osteoclast. Alternatively, such an inhibitory RANKL mimic may be generated deliberately, via modifications within the core jellyroll that may inhibit trimerization of the RANKL mimic, resulting in monomeric RANKL mimics that compete with endogenous trimeric RANKL and decrease RANK pathway signaling.

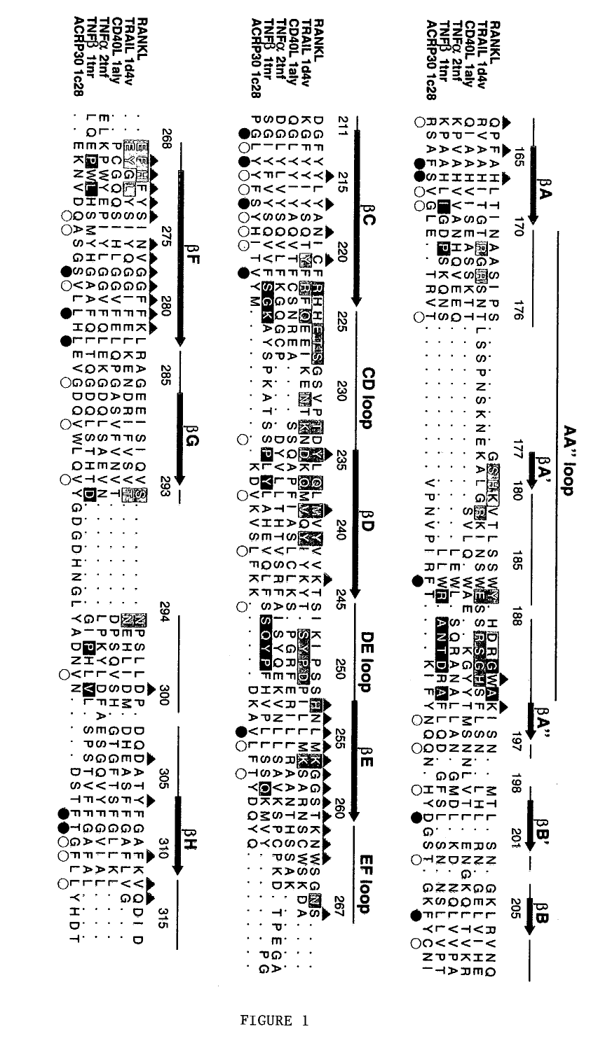

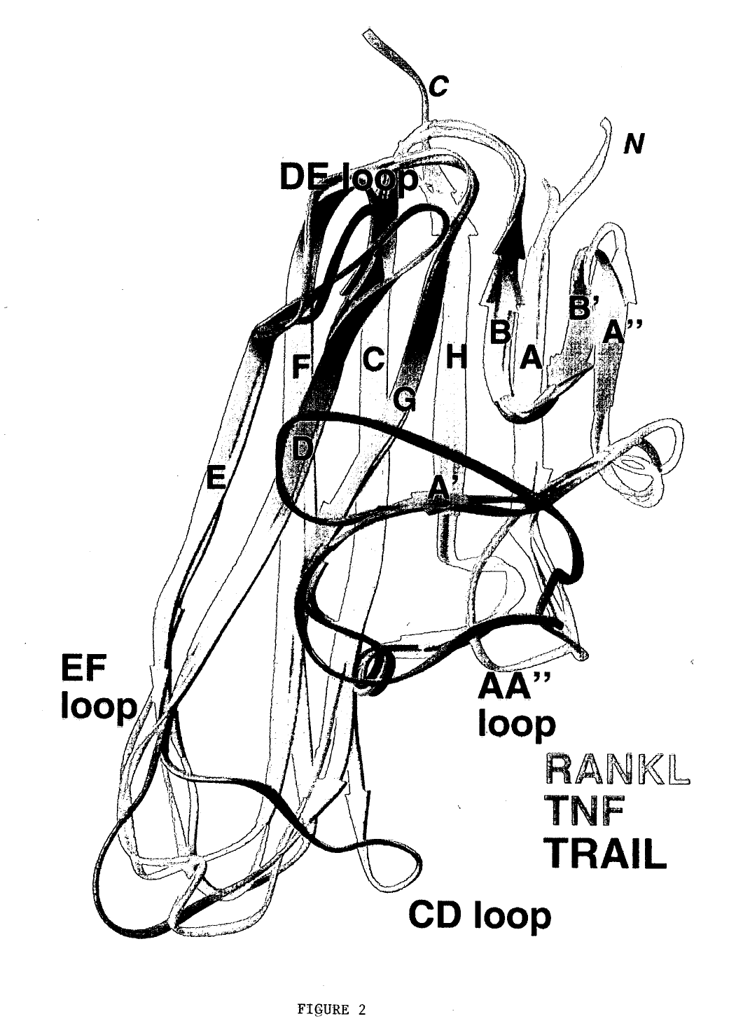

The external loops of the polypeptides encoded by the polynucleotides provided herein are substituted with external surface loops of RANKL by utilizing site directed mutagenesis. Initially, the polypeptide or a protein encoded by a desired polynucleotide is sequence aligned with RANKL protein by utilizing any of the sequence aligning programs known in the art. For example, see FIG. 1, which shows the alignment of RANKL with TRAIL, CD40L (CD40 ligand), TNFα, TNFβ, and ACRP30 (adipocyte complement-related protein). The sequence alignment suggests the identity of β strands that are significantly homologous and the identity of non-homologous external surface loops. This information, when corroborated by structural determination using methods known to those skilled in the art, for example, X-ray crystallography, NMR (nuclear magnetic resonance), or solution spectroscopy, allows to determine the position of the external loops in the DNA sequences.

Even in the absence of said structural information, it is possible to place the unique loops encoded by the polynucleotide of SEQ ID NO: 1 or SEQ ID NO: 51 onto proteins, including but not limited to those of the TNF superfamily, by methods and strategies known to those skilled in the art. The DNA sequences are manipulated to encode TNF family ligands comprising RANKL external surface loops. Such manipulation may be achieved by methods known to those skilled in the art, including by utilizing, e.g., a Quick-Change™ XL Site-Directed Mutagenesis Kit, available from Stratagene (http://www.stratagene.com). Briefly, the polynucleotide sequence encoding TNF superfamily member is introduced into a plasmid of choice by any of the methods known in the art. The plasmid is then denatured and mixed with mutagenic primers encoding for an external surface loop of RANKL, allowing the primers to anneal to the denatured plasmid. The reaction is temperature cycled to allow extension of the primers and generation of nicked circular strands. The parental DNA strands are then digested, and double-stranded nicked DNA molecules are transformed into XL-10 Gold E. coli which repair the nicks and allow expression of novel DNA sequences containing an external surface loop of RANKL. In order to substitute multiple loops, sets of primers encoding such loops are created and annealed to a desired polynucleotide sequence as described above until all of the loops have been substituted. Other methods of site-directed mutagenesis exist in the art, and a skilled artisan can easily perform such methods. In this way, the RANKL external surface loops may be placed on any suitable protein, including proteins not in the TNF family but having a long serum half-life, such as albumin. Alternatively, RANKL external surface loops may be chemically attached to such carrier proteins using methods standard in the art, including but not limited to disulfide bridging or other means of crosslinking.

The recombinant polynucleotides of the present invention can be used as cloning or expression vectors although other uses are possible. A cloning vector is a self-replicating DNA molecule that serves to transfer a DNA segment into a host cell. The three most common types of cloning vectors are bacterial plasmids, phages, and other viruses. An expression vector is a cloning vector designed so that a coding sequence inserted at a particular site will be transcribed and translated into a protein.

Both cloning and expression vectors contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences. Various bacterial and viral origins of replication are well known to those skilled in the art and include, but are not limited to the pBR322 plasmid origin, the 2μ plasmid origin, and the SV40, polyoma, adenovirus, VSV and BPV viral origins.

The polynucleotide sequences of the present invention may be used to produce proteins by the use of recombinant expression vectors containing the sequences. Suitable expression vectors include chromosomal, non-chromosomal and synthetic DNA sequences, for example, SV 40 derivatives; bacterial plasmids; phage DNA; baculovirus; yeast plasmids; vectors derived from combinations of plasmids and phage DNA; and viral DNA such as vaccinia, adenovirus, fowl pox virus, retroviruses, and pseudorabies virus. Preferably, a bacterial plasmid such as pGEX-6P-1, available from Amersham Pharmacia Biotech, Piscataway, N.J. is utilized. However, any other vector that is replicable and viable in the host may be used.

The nucleotide sequence of interest may be inserted into the vector by a variety of methods. In the most common method the sequence is inserted into an appropriate restriction endonuclease site(s) using procedures commonly known to those skilled in the art and detailed in, for example, Sambrook, et al., Molecular Cloning, A Laboratory Manual, 2nd ed., Cold Spring Harbor Laboratory Press, (1989) and Ausubel, et al., Short Protocols in Molecular Biology, 3rd ed., John Wiley & Sons (1995).

In an expression vector, the sequence of interest is operably linked to a suitable expression control sequence or promoter recognized by the host cell to direct mRNA synthesis. Promoters are untranslated sequences located generally 100 to 1000 base pairs (bp) upstream from the start codon of a structural gene that regulate the transcription and translation of nucleic acid sequences under their control. Promoters are generally classified as either inducible or constitutive. Inducible promoters are promoters that initiate increased levels of transcription from DNA under their control in response to some change in the environment, e.g., the presence or absence of a nutrient or a change in temperature. Constitutive promoters, in contrast, maintain a relatively constant level of transcription. In addition, useful promoters can also confer appropriate cellular and temporal specificity. Such promoters include those that are developmentally-regulated or organelle-, tissue- or cell-specific.

A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading frame. Linking is achieved by blunt end ligation or ligation at restriction enzyme sites. If suitable restriction sites are not available, then synthetic oligonucleotide adapters or linkers can be used as is known to those skilled in the art (Sambrook, et al., Molecular Cloning, A Laboratory Manual, 2nd ed., Cold Spring Harbor Laboratory Press, (1989) and Ausubel, et al., Short Protocols in Molecular Biology, 3rd ed., John Wiley & Sons (1995)).

Common promoters used in expression vectors include, but are not limited to, CMV promoter, LTR or SV40 promoter, the E. coli lac or trp promoters, and the phage lambda PL promoter. Other promoters known to control the expression of genes in prokaryotic or eukaryotic cells can be used and are known to those skilled in the art. Expression vectors may also contain a ribosome binding site for translation initiation, and a transcription terminator. The vector may also contain sequences useful for the amplification of gene expression.

Expression and cloning vectors can and usually do contain a selection gene or selection marker. Typically, this gene encodes a protein necessary for the survival or growth of the host cell transformed with the vector. Examples of suitable markers include dihydrofolate reductase (DHFR) or neomycin or hygromycin B resistance for eukaryotic cells and tetracycline, ampicillin, or kanamycin resistance for E. coli.

In addition, expression vectors can also contain marker sequences operatively linked to a nucleotide sequence for a protein that encode an additional protein used as a marker. The result is a hybrid or fusion protein comprising two linked and different proteins. The marker protein can provide, for example, an immunological or enzymatic marker for the recombinant protein produced by the expression vector. In a preferred embodiment of the present invention, alkaline phosphatase (AP), green fluorescence protein (GFP), myc, histidine tag (His) and hemagglutinin (HA) are used as markers.

Additionally, the end of the polynucleotide can be modified by the addition of a sequence encoding an amino acid sequence useful for purification of the protein produced by affinity chromatography. Various methods have been devised for the addition of such affinity purification moieties to proteins. Representative examples can be found in U.S. Pat. Nos. 4,703,004, 4,782,137, 4,845,341, 5,935,824, and 5,594,115, each of which is incorporated by reference in its entirety. Any method known in the art for the addition of nucleotide sequences encoding purification moieties can be used, for example those contained in Innis, et al., PCR Protocols, Academic Press (1990) and Sambrook, et al., Molecular Cloning, 2nd ed., Cold Spring Harbor Laboratory Press (1989). In one preferred aspect, glutathione-S-transferase (GST) is used to allow affinity purification of polypeptides of the present invention. In a more preferred embodiment, a polynucleotide sequence encoding the GST moiety is added to the 5′ end of the nucleotide. GST or another moiety such as a leucine zipper, SAM, or other domain may be added to induce oligomerization, to decrease the rate of internalization, or to otherwise mimic the compounds taught in U.S. application Ser. No. 60/277,855, filed Mar. 22, 2001.

More particularly, the present invention includes recombinant constructs comprising the modified polynucleotide sequences of the present invention. The constructs can include a vector, such as a plasmid or viral vector, into which the sequence of the present invention has been inserted, either in the forward or reverse orientation. The recombinant construct further comprises regulatory sequences, including for example, a promoter operatively linked to the sequence. Large numbers of suitable vectors and promoters are known to those skilled in the art and are commercially available. In one preferred embodiment, pGEX-6P-11 vectors are used. It will be understood by those skilled in the art, however, that other plasmids or vectors may be used as long as they are replicable and viable or expressing the encoded protein in the host.

The polynucleotide sequences of the present invention can also be part of an expression cassette that at a minimum comprises, operably linked in the 5′ to 3′ direction, a promoter, a polynucleotide of the present invention, and a transcriptional termination signal sequence functional in a host cell. The promoter can be of any of the types discussed herein, for example, a tissue specific promoter, a developmentally regulated promoter, and an organelle specific promoter. The expression cassette can further comprise an operably linked targeting sequence, transit or secretion peptide coding region capable of directing transport of the protein produced. The expression cassette can also further comprise a nucleotide sequence encoding a selectable marker and a purification moiety.

A further embodiment of the present invention relates to transformed host cells containing the constructs comprising the polynucleotide sequence of the present invention. The host cell can be a higher eukaryotic cell, such as a mammalian cell (including, without limitation Chinese Hamster Ovary (CHO) or COS cell lines), or a lower eukaryotic cell such as an insect cell or a yeast cell, or the host can be a prokaryotic cell such as a bacterial cell. In a preferred embodiment, a host cell is protease-deficient BL21 (DE3) Escherichia coli. Introduction of the construct into the host cell can be accomplished by a variety of methods including calcium phosphate transfection, DEAE-dextran mediated transfection, polybrene mediated transfection, protoplast fusion, liposome mediated transfection, direct microinjection into the nuclei, biolistic (gene gun) devices, scrape loading, and electroporation.

The term protein also includes forms of the RANKL mimic to which one or more substituent groups have been added. A substituent is an atom or group of atoms that is introduced into a molecule by replacement of another atom or group of atoms. Such groups include, but are not limited to lipids, phosphate groups, sugars and carbohydrates. Thus, the term protein includes, for example, lipoproteins, glycoproteins, phosphoproteins and phospholipoproteins.

The present invention also includes methods for the production of the RANKL mimic from cells transformed with the modified polynucleotide sequences of the present invention. Proteins can be expressed in mammalian cells, plant cells, insect cells, yeast, bacteria, bacteriophage, or other appropriate host cells. Host cells are genetically transformed to produce the protein of interest by introduction of an expression vector containing the nucleic acid sequence of interest. The characteristics of suitable cloning vectors and the methods for their introduction into host cells have been previously discussed. Alternatively, cell-free translation systems can also be employed using RNA derived from the DNA of interest. Methods for cell free translation are known to those skilled in the art. (Davis, et al., Basic Methods in Molecular Biology, Elsevier Science Publishing (1986); Ausubel, et al., Short Protocols in Molecular Biology, 2nd ed., John Wiley & Sons (1992)). In the preferred embodiment, host cells are HEK 293 cells or 293T cells (American Type Culture Collection).

Host cells are grown under appropriate conditions to a suitable cell density. If the sequence of interest is operably linked to an inducible promoter, the appropriate environmental alteration is made to induce expression. If the protein accumulates in the host cell, the cells are harvested by, for example, centrifugation or filtration. The cells are then disrupted by physical or chemical means to release the protein into the cell extract from which the protein can be purified. If the host cells secrete the protein into the medium, the cells and medium are separated and the medium retained for purification of the protein.

Larger quantities of protein can be obtained from cells carrying amplified copies of the sequence of interest. In this method, the sequence is contained in a vector that carries a selectable marker and transfected into the host cell or the selectable marker is co-transfected into the host cell along with the sequence of interest. Lines of host cells are then selected in which the number of copies of the sequence have been amplified. A number of suitable selectable markers will be readily apparent to those skilled in the art. For example, the dihydrofolate reductase (DHFR) marker is widely used for co-amplification. Exerting selection pressure on host cells by increasing concentrations of methotrexate can result in cells that carry up to 1000 copies of the DHFR gene.

Proteins recovered can be purified by a variety of commonly used methods, including, but not limited to, ammonium sulfate precipitation, immunoprecipitation, ethanol or acetone precipitation, acid extraction, ion exchange chromatography, size exclusion chromatography, affinity chromatography, high performance liquid chromatography, electrophoresis, and ultra filtration. If required, protein refolding systems can be used to complete the configuration of the protein. Preferably, the proteins are purified by affinity chromatography.

In a preferred embodiment of the invention, a method of preventing or inhibiting bone loss or of enhancing bone formation is provided by administering compositions comprising polypeptides of the present invention. The bone forming or anti-resorptive compositions of the present invention may be utilized by providing an effective amount of such compositions to a subject in need thereof.

For use for treatment of animal subjects, the compositions of the invention can be formulated as pharmaceutical or veterinary compositions. Depending on the subject to be treated, the mode of administration, and the type of treatment desired, e.g., prevention, prophylaxis, therapy; the compositions are formulated in ways consonant with these parameters. A summary of such techniques is found in Remington's Pharmaceutical Sciences, latest edition, Mack Publishing Co., Easton, Pa.

The administration of the compositions of the present invention may be pharmacokinetically and pharmacodynamically controlled by calibrating various parameters of administration, including the frequency, dosage, duration mode and route of administration. Thus, in one embodiment bone mass formation is achieved by administering a bone forming composition in a non-continuous, intermittent manner, such as by daily injection and/or ingestion. In another embodiment, bone resorption is inhibited by administering an anti-resorptive in a continuous or intermittent manner. Variations in the dosage, duration and mode of administration may also be manipulated to produce the activity required.

For administration to animal or human subjects, the dosage of the compounds of the invention is typically 0.01-100 mg/kg. However, dosage levels are highly dependent on the nature of the disease or situation, the condition of the patient, the judgment of the practitioner, and the frequency and mode of administration. If the oral route is employed, the absorption of the substance will be a factor effecting bioavailability. A low absorption will have the effect that in the gastro-intestinal tract higher concentrations, and thus higher dosages, will be necessary.

It will be understood that the appropriate dosage of the substance should suitably be assessed by performing animal model tests, wherein the effective dose level (e.g. ED₅₀) and the toxic dose level (e.g. TD₅₀) as well as the lethal dose level (e.g LD₅₀ or LD₁₀ ) are established in suitable and acceptable animal models. Further, if a substance has proven efficient in such animal tests, controlled clinical trials should be performed.

In general, for use in treatment, the compounds of the invention may be used alone or in combination with other compositions for the treatment of bone loss. Such compositions include anti-resorptives such as a bisphosphonate, a calcitonin, a calcitriol, an estrogen, SERM's and a calcium source, or a bone formation agent like parathyroid hormone or its derivative, a bone morphogenic protein, osteogenin, NaF, or a statin. See U.S. Pat. No. 6,080,779 incorporated herein by reference in its entirety. Depending on the mode of administration, the compounds will be formulated into suitable compositions.

Formulations may be prepared in a manner suitable for systemic administration or for topical or local administration. Systemic formulations include, but are not limited to those designed for injection (e.g., intramuscular, intravenous or subcutaneous injection) or may be prepared for transdermal, transmucosal, rectal, nasal, or oral administration. The formulation will generally include a diluent as well as, in some cases, adjuvants, buffers, preservatives and the like. For oral administration, the compositions can be administered also in liposomal compositions or as microemulsions. Suitable forms include syrups, capsules, tablets, as is understood in the art. For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

The compositions of the present invention may also be administered locally to sites in patients, both human and other vertebrates, such as domestic animals, rodents and livestock, where bone formation and growth are desired using a variety of techniques known to those skilled in the art. For example, these may include sprays, lotions, gels or other vehicles such as alcohols, polyglycols, esters, oils and silicones. Such local applications include, for example, at a site of a bone fracture or defect to repair or replace damaged bone. Additionally, a bone forming composition may be administered, e.g., in a suitable carrier, at a junction of an autograft, allograft or prosthesis and native bone to assist in binding of the graft or prosthesis to the native bone.

In another aspect, a method of enhancing processes of bone formation involves administering an effective amount of a polypeptide that binds to RANK on osteoblasts or related cells under conditions sufficient for RANK activation. Alternatively, the compound may interact with but not activate RANK; in osteoclasts this may inhibit binding of native RANKL and result in decreased bone resorption. For instance, binding to RANK is determined by performing an assay such as, e.g., a binding assay between a desired compound and RANK. In one aspect, this is done by contacting said compound to RANK and determining its dissociation rate. Numerous possibilities for performing binding assays are well known in the art. The indication of a compound's ability to bind to RANK is determined, e.g., by a dissociation rate, and the correlation of binding activity and dissociation rates is well established in the art. For example, the assay may be performed by radio-labeling a reference compound, e.g., RANKL or a RANKL fragment, analog or derivative that binds RANK, including but not limited to an external surface loop of RANKL, with ¹²⁵I and incubating it with RANK in 1.5 ml tubes. Test compounds are then added to these reactions in increasing concentrations. After optimal incubation, the RANK/compound complexes are separated, e.g., with chromatography columns, and evaluated for bound ¹²⁵I-labeled peptide with (gamma) γ counter. The amount of the test compound necessary to inhibit 50% of the reference compound's binding is determined. These values are then normalized to the concentration of unlabeled reference compound's binding (relative inhibitory concentration (RIC)⁻¹=concentrationₜₑₛₜ/concentration_{reference}). A small RIC⁻¹ value indicates strong relative binding, whereas a large RIC⁻¹ value indicates weak relative binding. See, for example, Latek, et al., Proc. Natl. Acad Sci. USA, Vol. 97, No. 21, pp. 11460-11465, 2000. Of course, high throughput assays may be used for screening of molecules that bind RANK as well.

Biological activity may alternatively be determined by measuring the activity of downstream elements of the RANK pathway as taught in U.S. applications Ser. No. 60/329,231 filed Oct. 12, 2001 and 60/328,876 filed Oct. 12, 2001. Depending on the substitutions in the compounds of the present invention, RANK and its downstream effectors on either osteoblasts or osteoclasts may be activated or inhibited or both and lead either to decreased bone resorption or formation of bone.

A general protocol for treatment of osteoblasts or related cells with a compound/peptide is well established in the art. See, for instance, Wyatt, et al., BMC Cell Biology, 2:14, 2001. A cell line of choice in this article was MC3T3-E1, which has been used as an in vitro model of osteoblastic differentiation and maturation. The treatment of cells, in this case with BMP-2, was performed in the following manner. The cells were plated at 5000/cm² in plastic 25 cm² culture flasks in α-MEM supplemented with 5% fetal bovine serum, 26 mM NaHCO₃, 2 mM glutamine, 100 u/ml penicillin, and 100 μg/ml streptomycin, and grown in humidified 5% CO₂/95% air at 37° C. Cells were passaged every 3-4 days after releasing with 0.002% pronase E in PBS. The cells in treatment groups were grown for 24 hours, then incubated with BMP-2 (50 ng/ml) dissolved in PBS containing 4 mM HCl and 0.1% bovine serum albumin (BSA) at 37° C. for 24 and 48 hours. Control groups received equal volumes of vehicles only. It is to be noted that the conditions used will vary according to the cell lines and compound used, their respective amounts, and additional factors such as plating conditions and media composition. Such adjustments are readily determinable by one skilled in this art.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications and patent applications cited in this specification are herein incorporated by reference as if each individual publication or patent application was specifically and individually indicated to be incorporated by reference. The following examples illustrate the invention, but are not to be taken as limiting the various aspects of the invention so illustrated.

### EXAMPLES

### Example 1

AP activity following RANKL exposure in osteoblasts. Primary calvarial osteoblasts were cultured in MEM containing 15% FBS, 50 μM ascorbic acid, and 10 mM β-glycerophosphate. Cells were maintained at 37° C., with daily replenishment of media and cytokines. Osteoblast alkaline phosphatase (AP) activity, a direct measure of osteoblast differentiation and function, was quantitated by addition of a colorimetric substrate, 5.5 mM p-nitrophenyl phosphate. The cells were then exposed to RANKL, administered in different regimens. Pulsatile exposure to 50 ng/ml GST-RANKL was provided at 1, 3, 6, 8, or 24 hours of total exposure per 48-hour treatment window. After 4 such 48-hour treatments, AP activity was quantitated (±S.D.) and normalized to total protein levels.

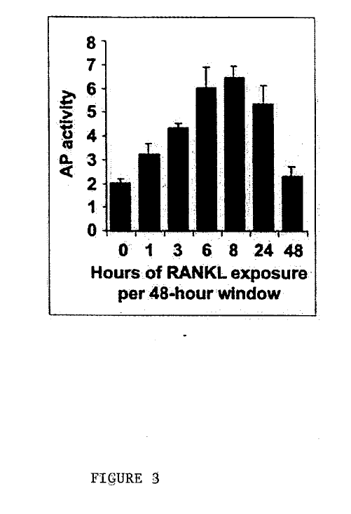

As can be seen from FIG. 3, the maximum anabolic effect was observed when GST-RANKL exposure was provided for an 8-hour treatment window, once every 48 hours. Thus, GST-RANKL induced increase in AP activity when administered in an intermittent fashion.

### Example 2

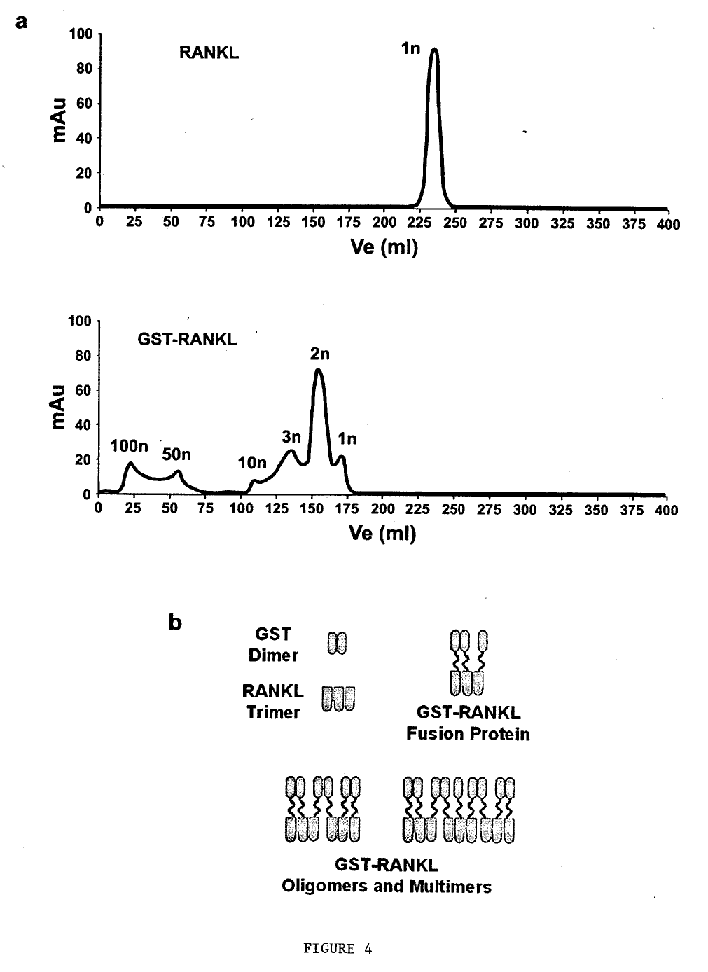

Oligomerization of GST-RANKL. GST-RANKL was subjected to proteolysis to isolate the cleaved RANKL fragment from its GST fusion partner. Briefly, GST-RANKL was incubated with the type-14 human rhinovirus 3C protease (Amersham Pharmacia Biotech) for 4 hours at 4° C. in 50 mM Tris-HCl, pH 7.0, 150 mM NaCl, 10 mM EDTA, and 1 mM DTT. Uncleaved fusion protein and GST-tagged protease were removed by passage over a glutathione affinity matrix. All purified recombinant proteins were assayed for endotoxin contamination by limulus amoebocyte lysate assay (Bio Whittaker), and analyzed by mass spectrometry to confirm identity. Both GST-RANKL and cleaved RANKL were dialyzed against physiologic salt and pH, and fractionated by gel filtration in Superose-6 26/60 using an AKTA explorer chromatography system (Amersham Pharmacia). Elution volumes were calibrated to molecular weight using the following standards: ribonuclease A (13,700), chymotrypsinogen A (25,000), ovalbumin (43,000), bovine serum albumin (67,000), aldolase (158,000), catalase (232,000), ferritin (440,000), thyroglobulin (669,000), and blue dextran 2000 (2,000,000). Fractions containing protein from different elution volumes were subjected to Western analysis using a monoclonal anti-GST primary antibody. As FIG. 4(a) shows, cleaved RANKL migrated as a single trimeric species (1n), whereas GST-RANKL migrated as a polydisperse mixture of non-covalently associated mono-trimeric (1n) and oligomeric (2-100 n) under dynamic equilibrium. Crystallographic evidence has established that GST possesses an innate tendency to dimerize, while RANKL spontaneously trimerizes. A single GST-RANKL trimer, consisting of 3 RANKL molecules and 3 GST molecules, thus contains a free GST that is not bound to a neighboring GST, resulting in a 3:2 stoichiometry that engenders a propensity to oligomerize. High-order, branched oligomers form when the GST of a given GST-RANKL trimer forms a dimer with the GST from a neighboring GST-RANKL trimer (see FIG. 4(b)).

### Example 3

Internalization of GST-RANKL. Primary murine osteoblasts were maintained in α-MEM containing 10% fetal bovine serum, and cultured in MEM containing 15% FBS, 50 μM ascorbic acid, and 10 mM β-glycerophosphate for differentiation. Cells were maintained at 37° C. in a humidified atmosphere containing 6% CO₂, with daily replenishment of media and cytokines. Primary murine osteoblasts were cultured on coverslips in α-MEM containing 10% fetal bovine serum and treated with GST-RANKL or cleaved RANKL for the indicated times. For phospholipid membrane staining, cells were incubated for 20 minutes with Vybrant DiI lipophilic carbocyanine membrane fluorescent stain (Molecular Probes). Cells were fixed in 4% paraformaldehyde, permeabilized with 0.1 % Triton-X, blocked with 1% BSA/0.2% nonfat dry milk in PBS, and stained for RANK with a polyclonal anti-RANK antibody. Serial optical sections were obtained using a Radiance2100 laser scanning confocal microscope (BioRad). Microscope settings were calibrated to black level values using cells stained with an isotypic Ig control. GST-RANKL was cleaved as described in Example 2.

Primary osteoblasts in culture were exposed to 5 nM cleaved RANKL or GST-RANKL. At the indicated times, the cell surface was stained with a lipophilic fluorescent dye, and RANK was stained with an anti-RANK antibody. Confocal microscopy was employed to localize RANK (green fluorescence) and the cell surface (red fluorescence). On the merged images, colocalization of RANK and the cell surface appears yellow (overlap of green and red fluorescence). GST-RANKL:RANK complexes remain on the cell surface for at least one hour, corresponding to the sustained intracellular RANK signaling. In contrast, cleaved RANKL-RANK complexes are completely internalized within one hour, correlating to the absence of cleaved RANKL-induced RANK signaling at that time. Results are shown in FIG. 5.

### Example 4

Loop Substitution. Using methods well known in the art, nucleotide sequences encoding various loops of RANKL may replace surface loops of TNF superfamily proteins. The resulting RANKL mimic is recombinantly expressed as discussed herein and well known in the art. For illustrative purposes only, among many possibilities is replacement of surface loops of TNFα with corresponding loops of RANKL. Specifically, using a full length nucleotide sequence that encodes human TNFα (such as that of accession number NM_{—}000594) as a backbone, AA″ replacement is accomplished by substituting nucleotides 368-418 of NM_{—}000594 which encode the AA″ loop (SEQ ID NO: 47) of TNFα with nucleotides 639-710 of human RANKL (SEQ ID NO: 51) which encode the AA″ loop (SEQ ID NO: 43 or SEQ ID NO: 52) of RANKL. CD loop replacement is accomplished by substituting nucleotides 512-526 of NM_{—}000594 which encode the CD loop (SEQ ID NO: 48) of TNFα with nucleotides 801-830 of SEQ ID NO 51 which encode the CD loop (SEQ ID NO: 44) of RANKL. DE loop replacement is accomplished by substituting nucleotides 563-583 of NM_{—}000594 which encode the DE loop (SEQ ID: 49) of TNFα with nucleotides 864-884 of SEQ ID NO: 51 which encode the DE loop (SEQ ID NO: 45) of RANKL. EF loop replacement is accomplished by substituting nucleotides 611-652 of NM_{—}000594 which encode the EF loop (SEQ ID NO: 50) of TNFα with nucleotides 912-938 of SEQ ID NO 51 which encode the EF loop (SEQ ID NO: 46) of RANKL. Some or all of these substitutions may be undertaken to create a human RANKL mimic from a TNFα backbone.

What is claimed is:

1. A RANKL mimic comprising a core, at least one external loop, wherein the sequence of the mimic core comprises the sequence of the core of a non-RANKL TNF superfamily member, and wherein the sequence of at least one external loop is the sequence of the homologous RANKL external loop having essentially the sequence of SEQ ID NO: 43, SEQ ID NO: 44, SEQ ID NO: 45, SEQ ID NO: 46 or SEQ ID NO: 52.

2. The RANKL mimic of claim 1, wherein the sequence of each external loop is the sequence of the homologous loop of RANKL.

3. A polynucleotide comprising a coding sequence that encodes a RANKL mimic comprising a core, at least one external loops, wherein the sequence of the mimic core comprises the sequence of the core of a non-RANKL TNF superfamily member, wherein the sequence of at least one external loop is the sequence of the homologous RANKL external loop having essentially the sequence of SEQ ID NO: 43, SEQ ID NO: 44, SEQ ID NO: 45, SEQ ID NO: 46 or SEQ ID NO: 52.

4. The polynucleotide of claim 3, which further comprises a promoter operably linked to the coding sequence.

5. A recombinant cell expressing the polynucleotide of claim 4.

6. The recombinant cell of claim 5, wherein the cell is a eukaryotic cell.

7. A monomeric RANKL mimic comprising a core and at least one external loop, wherein the sequence of the core comprises the sequence of the core of a non-RANKL TNF superfamily member modified in the trimerizing region, such that it is unable to form trimers, the sequence of at least one external loop is the sequence of the homologous RANKL external loop having essentially the sequence of SEQ ID NO: 43, SEQ ID NO: 44, SEQ ID NO: 45, SEQ ID NO: 46 or SEQ ID NO: 52.

8. The monomeric RANKL mimic of claim 7, wherein the sequence of each external loop is the sequence of the homologous loop of RANKL.

9. A polynucleotide comprising a coding sequence that encodes a monomeric RANKL mimic comprising a core and at least one external loop, wherein the sequence of the core comprises the sequence of the core of a non-RANKL TNF superfamily member modified in the regions mediating trimerization, such that they no longer homotrimerize, the sequence of at least one external loop is the sequence of the homologous RANKL external loop having essentially the sequence of SEQ ID NO: 43, SEQ ID NO: 44, SEQ ID NO: 45 or SEQ ID NO: 46.

10. The polynucleotide of claim 9, which further comprises a promoter operably linked to the coding sequence.

11. A recombinant cell expressing the polynucleotide of claim 10.

12. The recombinant cell of claim 11, wherein the cell is a eukaryotic cell.

13. The recombinant RANKL mimic of claim 1, wherein the TNF superfamily member is selected from the group consisting of TRAIL, CD40L, TNF-alpha, TNF-beta, and ACRP30.

14. The recombinant RANKL mimic of claim 7, wherein the TNF superfamily member is selected from the group consisting of TRAIL, CD40L, TNF-alpha, TNF-beta, and ACRP30.

15. The polynucleotide sequence of claim 3, wherein the TNF superfamily member is selected from the group consisting of TRAIL, CD40L, TNF-alpha, TNF-beta, and ACRP30.

16. The polynucleotide sequence of claim 9, wherein the TNF superfamily member is selected from the group consisting of TRAIL, CD40L, TNF-alpha, TNF-beta, and ACRP30.

17. A recombinant RANKL mimic in which the TNF superfamily member is RANKL or other TNF superfamily member wherein the oligomerizing DE loop is that of TALL-1, and at least one of the remaining external loops are the AA″, EF, and CD loops of RANKL.

18. A polynucleotide encoding the RANKL mimic of claim 17.

19. An oligomeric RANKL mimic comprising a core, at least one external loop and an oligomerizing domain, wherein the sequence of the mimic core comprises the sequence of the core of a non-RANKL TNF superfamily member, and wherein the sequence of at least one external loop is the sequence of the homologous RANKL external loop having essentially the sequence of SEQ ID NO: 43, SEQ ID NO: 44, SEQ ID NO: 45, SEQ ID NO: 46 or SEQ ID NO: 52.

20. The oligomeric RANKL mimic of claim 19, wherein the sequence of each external loop is the sequence of the homologous loop of RANKL.

21. A polynucleotide; comprising a coding sequence that encodes an oligomeric RANKL mimic comprising a core, at least one external loop and an oligomerizing domain, wherein the sequence of the mimic core comprises the sequence of the core of a non-RANKL TNF superfamily member, and wherein the sequence of at least one external loop is the sequence of the homologous RANKL external loop having essentially the sequence of SEQ ID NO: 43, SEQ ID NO: 44, SEQ ID NO: 45, SEQ ID NO: 46 or SEQ ID NO: 52.

22. The polynucleotide of claim 21, further comprising a promoter operably linked to the coding sequence.

23. A recombinant cell expressing the polynucleotide of claim 22.

24. The recombinant cell of claim 23, wherein the cell is a eukaryotic cell.

25. The recombinant RANKL mimic of claim 19, wherein the TNF superfamily member is selected from the group consisting of TRAIL, CD40L, TNF-alpha, TNF-beta, and ACRP30.

26. The polynucleotide sequence of claim 21, wherein the TNF superfamily member is selected from the group consisting of TRAIL, CD40L, TNF-alpha, TNF-beta, and ACRP30.

27. A recombinant RANKL mimic in which the TNF superfamily member is RANKL or other TNF superfamily member wherein the oligomerizing DE loop is that of TALL-1, and at least one of the remaining external loops are the AA″, EF, or CD loops of RANKL.

28. A polynucleotide encoding the oligomerizing RANKL mimic of claim 27.

29. The RANKL mimic of claim 19, wherein the oligomerizing domain is operably linked to the amino terminus.

30. The RANKL mimic of claim 29, wherein the oligomerizing domain is glutathione S-transferase.

31. The RANKL mimic of claim 19, wherein the oligomerizing domain is operably linked to the carboxy terminus.

32. The RANKL mimic of claim 19,-wherein the oligomerizing domain is internally linked.

33. The RANKL mimic of claim 29, wherein the oligomerizing domain is glutathione S-transferase.

34. A polynucleotide encoding the RANKL mimic of claim 29.

35. A polynucleotide encoding the RANKL mimic of claim 30.

36. A polynucleotide encoding the RANKL mimic of claim 31.

37. A polynucleotide encoding the RANKL mimic of claim 32.


SEQUENCE LISTING (52 sequences)

SEQ ID NO: 1 - DNA, 1823 bp, Homo sapiens
  ref <308>: DATABASE ACCESSION NUMBER: NCBI/AF013171
  ref <309>: DATABASE ENTRY DATE: 1997-09-19
  ref <313>: RELEVANT RESIDUES: (1)..(1823)
  ref <308>: DATABASE ACCESSION NUMBER: NCBI/NM_033012.2
  ref <309>: DATABASE ENTRY DATE: 2002-07-31
  ref <313>: RELEVANT RESIDUES: (1)..(1823)
  ref <308>: DATABASE ACCESSION NUMBER: NCBI/AF053712.1
  ref <309>: DATABASE ENTRY DATE: 1998-05-09
  ref <313>: RELEVANT RESIDUES: (1)..(1823)
  ref <308>: DATABASE ACCESSION NUMBER: NCBI/AF019047.1
  ref <309>: DATABASE ENTRY DATE: 1997-11-22
  ref <313>: RELEVANT RESIDUES: (1)..(1823)
cagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcgggggtggggtaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggcccaacggtacacgactcagtatccatgctcttgaccttgtagagaacacgcgtatttacagccagtgggagatgttagactcatggtgtgttacacaatggtttttaaattttgtaatgaattcctagaattaaaccagattggagcaattacgggttgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatctgtcaagagtagttatataattgttgaacaggtgtttttccacaagtgccgcaaattgtacctttttttgtttttttcaaaatagaaaagttattagtggtttatcagcaaaaaagtccaattttaatttagtaaatgttatcttatactgtacaataaaaacattgcctttgaatgttaattttttggtacaaaagtcgacggccgc

SEQ ID NO: 2 - DNA, 1769 bp, Homo sapiens
  ref <308>: DATABASE ACCESSION NUMBER: NCBI/NM_003810.2
  ref <309>: DATABASE ENTRY DATE: 2002-10-07
  ref <313>: RELEVANT RESIDUES: (1)..(1769)
  ref <308>: DATABASE ACCESSION NUMBER: NCBI/U57059.1
  ref <309>: DATABASE ENTRY DATE: 1999-03-04
  ref <313>: RELEVANT RESIDUES: (1)..(1769)
  ref <308>: DATABASE ACCESSION NUMBER: NCBI/U37518.1
  ref <309>: DATABASE ENTRY DATE: 1996-01-06
  ref <313>: RELEVANT RESIDUES: (1)..(1769)
  ref <308>: DATABASE ACCESSION NUMBER: NCBI/BC032722.1
  ref <309>: DATABASE ENTRY DATE: 2002-06-27
  ref <313>: RELEVANT RESIDUES: (1)..(1769)
cctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagaaaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagcgcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtgcaaaaattagctgggtgtgttggcacatgcctgtagtcccagctacttgagaggctgaggcaggagaatcgtttgaacccgggaggcagaggttgcagtgtggtgagatcatgccactacactccagcctggcgacagagcgagacttggtttcaaaaaaaaaaaaaaaaaaaaacttcagtaagtacgtgttatttttttcaataaaattctattacagtatgtcaaaaaaaaaaaaaaaaaa

SEQ ID NO: 3 - DNA, 1803 bp, Homo sapiens
  ref <308>: DATABASE ACCESSION NUMBER: NCBI/X67878.1
  ref <309>: DATABASE ENTRY DATE: 1997-06-06
  ref <313>: RELEVANT RESIDUES: (1)..(1803)
  ref <308>: DATABASE ACCESSION NUMBER: NCBI/X68550.1
  ref <309>: DATABASE ENTRY DATE: 1993-06-30
  ref <313>: RELEVANT RESIDUES: (1)..(1803)
  ref <308>: DATABASE ACCESSION NUMBER: NCBI/NM_000074.1
  ref <309>: DATABASE ENTRY DATE: 2002-04-10
  ref <313>: RELEVANT RESIDUES: (1)..(1803)
  ref <308>: DATABASE ACCESSION NUMBER: NCBI/L07414.1
  ref <309>: DATABASE ENTRY DATE: 1993-04-27
  ref <313>: RELEVANT RESIDUES: (1)..(1803)
tgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacatt

SEQ ID NO: 4 - DNA, 1643 bp, Homo sapiens
  ref <308>: DATABASE ACCESSION NUMBER: NCBI/ X01394.1
  ref <309>: DATABASE ENTRY DATE: 1995-03-21
  ref <313>: RELEVANT RESIDUES: (1)..(1643)
  ref <308>: DATABASE ACCESSION NUMBER: NCBI/ BC028148.1
  ref <309>: DATABASE ENTRY DATE: 2002-05-01
  ref <313>: RELEVANT RESIDUES: (1)..(1643)
  ref <308>: DATABASE ACCESSION NUMBER: NCBI/ M35592.1
  ref <309>: DATABASE ENTRY DATE: 1993-04-27
  ref <313>: RELEVANT RESIDUES: (1)..(1643)
  ref <308>: DATABASE ACCESSION NUMBER: NCBI/ XM_165823.1
  ref <309>: DATABASE ENTRY DATE: 2002-08-01
  ref <313>: RELEVANT RESIDUES: (1)..(1643)
gcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaattgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgctt

SEQ ID NO: 5 - DNA, 1325 bp, Homo sapiens
  ref <308>: DATABASE ACCESSION NUMBER: NCBI/X01393.1
  ref <309>: DATABASE ENTRY DATE: 1993-07-12
  ref <313>: RELEVANT RESIDUES: (1)..(1325)
  ref <308>: DATABASE ACCESSION NUMBER: NCBI/NM_000595.2
  ref <309>: DATABASE ENTRY DATE: 2001-02-22
  ref <313>: RELEVANT RESIDUES: (1)..(1325)
  ref <308>: DATABASE ACCESSION NUMBER: NCBI/D12614.1
  ref <309>: DATABASE ENTRY DATE: 2000-02-01
  ref <313>: RELEVANT RESIDUES: (1)..(1325)
  ref <308>: DATABASE ACCESSION NUMBER: NCBI/D00102.1
  ref <309>: DATABASE ENTRY DATE: 2000-02-01
  ref <313>: RELEVANT RESIDUES: (1)..(1325)
gaggtttattgggcctcggtcctcctgcacctgctgcctggatccccggcctgcctgggcctgggccttggttctccccatgacaccacctgaacgtctcttcctcccaagggtgtgtggcaccaccctacacctcctccttctggggctgctgctggttctgctgcctggggcccaggggctccctggtgttggcctcacaccttcagctgcccagactgcccgtcagcaccccaagatgcatcttgcccacagcaccctcaaacctgctgctcacctcattggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctgtagaacttggaaaaatccagaaagaaaaaataattgatttcaagaccttctccccattctgcctccattctgaccatttcaggggtcgtcaccacctctcctttggccattccaacagctcaagtcttccctgatcaagtcaccggagctttcaaagaaggaattctaggcatcccaggggacccacactccctgaaccatccctgatgtctgtctggctgaggatttcaagcctgcctaggaattcccagcccaaagctgttggtcttgtccaccagctaggtggggcctagatccacacacagaggaagagcaggcacatggaggagcttgggggatgactagaggcagggaggggactatttatgaaggcaaaaaaattaaattatttatttatggaggatggagagagggaataatagaagaacatccaaggagaaacagagacaggcccaagagatgaagagtgagagggcatgcgcacaaggctgaccaagagagaaagaagtaggcatgagggatcacagggccccagaaggcagggaaaggctctgaaagccagctgccgaccagagccccacacggaggcatctgcaccctcgatgaagcccaataaacctcttttctctga

SEQ ID NO: 6 - DNA, 5307 bp, Homo sapiens
  misc_feature (4242)..(4242): n = a, t, c or g
  misc_feature (4471)..(4471): n = a, t, c or g
  misc_feature (4523)..(4523): n = a, t, c or g
  misc_feature (4529)..(4529): n = a, t, c or g
  misc_feature (4531)..(4531): n = a, t, c or g
  misc_feature (4545)..(4545): n = a, t, c or g
  ref <308>: DATABASE ACCESSION NUMBER: NCBI/NM_001399.1
  ref <309>: DATABASE ENTRY DATE: 2000-10-31
  ref <313>: RELEVANT RESIDUES: (1)..(5307)
  ref <308>: DATABASE ACCESSION NUMBER: NCBI/AF040628.1
  ref <309>: DATABASE ENTRY DATE: 1998-11-13
  ref <313>: RELEVANT RESIDUES: (1)..(5307)
  ref <308>: DATABASE ACCESSION NUMBER: NCBI/AF061189.1
  ref <309>: DATABASE ENTRY DATE: 1998-11-14
  ref <313>: RELEVANT RESIDUES: (1)..(5307)
attccctcggcgggccgagcctcccctctctcccgcccctcctcctccctttcccacccctcggagtagagctgcacatgcggctgctccctgctccgtcccgcccagccactgtcgcgcaggaacgggtccctgcagcccccagccgatggcaggacagtagccgcctgtcagaggtcgtgaacggctgaggcagacgcagcggctcccgggcctcaagagagtggatgtctccggaggccatgggctacccggaggtggagcgcagggaactcctgcctgcagcagcgccgcgggagcgagggagccagggctgcgggtgtggcggggcccctgcccgggcgggcgaagggaacagctgcctgctcttcctgggtttctttggcctctcgctggccctccacctgctgacgttgtgctgctacctagagttgcgctcggagttgcggcgggaacgtggagccgagtcccgccttggcggctcgggcacccctggcacctctggcaccctaagcagcctcggtggcctcgaccctgacagccccatcaccagtcaccttgggcagccgtcacctaagcagcagccattggaaccgggagaagccgcactccactctgactcccaggacgggcaccagatggccctattgaatttcttcttccctgatgaaaagccatactctgaagaagaaagtaggcgtgttcgccgcaataaaagaagcaaaagcaatgaaggagcagatggcccagttaaaaacaagaaaaagggaaagaaagcaggacctcctggacccaatggccctccaggacccccaggacctccaggaccccagggacccccaggaattccagggattcctggaattccaggaacaactgttatgggaccacctggtcctccaggtcctcctggtcctcaaggaccccctggcctccagggaccttctggtgctgctgataaagctggaactcgagaaaaccagccagctgtggtgcatctacagggccaagggtcagcaattcaagtcaagaatgatctttcaggtggagtgctcaatgactggtctcgcatcactatgaaccccaaggtgtttaagctacatccccgcagcggggagctggaggtactggtggacggcacctacttcatctatagtcaggtagaagtatactacatcaacttcactgactttgccagctatgaggtggtggtggatgagaagcccttcctgcagtgcacacgcagcatcgagacgggcaagaccaactacaacacttgctataccgcaggcgtctgcctcctcaaggcccggcagaagatcgccgtcaagatggtgcacgctgacatctccatcaacatgagcaagcacaccacgttctttggggccatcaggctgggtgaagcccctgcatcctagattccccccattttgcctctgtccgtgccccttccctgggtttgggagccaggactcccagaacctctaagtgctgctgtggagtgaggtgtattggtgttgcagccgcagagaaatgccccagtgttatttattccccagtgactccagggtgacaaggcctgcttgactttccagaatgaccttgagttaacaggacagttgatggagccccagggtttacatgaagcagaaccttctttggttccatgttgactgacttatggcatgactcttcaaccccgaggtccctgttgtcagatctattgtttgttgcactaaaatgaggatccagggcagcaggccagagaaagcaaaggtgcactccagactctgggggtggacatctgaccccaagggggctgctgctcctctcttgggtagggtagtggctggggtggagtgggaagkgagcattgcagcctaagaagaaggccagagagggaaaaggcaggtgcttttggcagagaccataagagaaacctgccaaggagcatccttggcagtgggaatgttctttctgctctatactgtggcctgcaggagggttggagtgctcttcccactccagctgacagccacaccgtggcagcttgctgggctttgggaagtttgctgtgctttggaacaatcacagggaatggccacaaacctgcccgcctaagaccctgaatccgtacttgggtcacatgactctcattttatttacagctgtgctccacactcagaaaattccctggggtcaccttctagttgcccccattcccagcctgactagaactcctgtcttctttctccatggagcctacctctgtctgagacaggtgcctaacctgggacctgtggtcatgtgagtctgggatattctttagcttacctgggcacagacagaattttccatttattaagcagtacagatgtttttcatccattcctaatcaaattctgtctggggacgaagggttggacgggatgacctccagaagtcccttcaatttctagtacctgtgactcttagccctcaccacagccttctaaattcccaaatcctagactgctcctgggcattagcaaggcagagcctttttacctggcctagaaagggcaaggggtgaggataggacagagggattttgttcaagtttgctgcaacccaagtggacgttaggccaggccttatctgaaaggccagcagctgatgctgtactaacccagtctttcttcactctggcttcaaaaagccacagcagagcattgtcaccgcaggtccccatgctgctcccctaaagccaggctcaggagaagccagtgtctaggcactgagcagggatctgccccctagttcaggtccaaattcaccttcccctaaaccccaagcttcccaacagatcatatggtaggaccctcgagagccttacttcaaagtgcctgggctcagcctggtttctgggtgctagatccagcccaaacctgggaaggccagccttgtacagtctgctcctcttgttcctgaaatgtgtttccttttcaggagatggggaataatttccttcaggcagctgaaattcaccaagaacagcgggtacttatttctcaagctgtgccttccctttctaagcaaccacactgcttggcccttcaagggtcagggtgagacgtgatgggctaggcctccgttgtctggttgctaatgacagccttgcaacccaaggtgaggtgaactccaggcatgtgtctggccctaactcctataaagtgcctcggacagtccgcagttgtagcagaaaccaacaagaaccactccttcatgtttggaaaataatttctcttgtattatctcctttgaagaaggcaaggctgataatatgacaaacatcattgtttagatgaggctcagagaggtagcactctcagagtgttttgaccagtttaagccgcagacctggagcttcagccaggtctgactccaaagctgttccattacaccacagcattgtgtggaatttgaggtctagagagaaccaataaaagtggtaattgggaactgaaatccttgagagttccggggagaaacccagagatgcctgatttcattcctcgatggtaatacccgtcctctcggctgccaggggctctgtggcaaaaagagtcagacatttctttggaaaacagcgaacagccttagagctcttgtgttcagaagaatcttcctggcacaatgttggagcagcaggcctctgggacccacagaacttgtggcctttatgttctttcacccatcctaggaaccagccaaccatcatgtgtagagcccctactgtgggcaaagtcctcctttcattaccctacagacagcttacaggagccagcctgcttcccacaactactagtgtgactccttatctctttccaccataccttagagactttgatactaccagggtctctcagggatggagggaagacctgaaagagaggactggttctgaggccagaaaggtgtgaggagagaggaggaaaagtcttcctaattgtgcccctaaagagcatcctgataccattctattctccagacatggaggggatgataaaggaaataggatctccactggacccttgattcattctgaaccctccaaaggaactctaagagggcgagggatgatgagggaagcaataggtagctggggagccctattgctgctaagtcattggcaaagtgcaaagcaatttactgatgagagaatgtggaaatagatgtgcagtttggaattatgttggtgtnaatttgccagaggaccaatgcttgcatggagaatggacgaggacatttgtgggcaagcagatgacagaggtttgaaggagaatggcatggcaggagtctctgccagttacttgggcttcaacagccaagctggcacaaaagacagctggcggaggctgctcggctactggttacctggagaagtagtatttgcctatttcccccttcatccatcctgagccaaatttcntttgctgaacaggaaagagcyaggaaccctggaggtaaacaaagactttgancctgtntnagtgtatgtgtttntgtaacttcctgtggagtgcaaatagattcagagaaatttagagctaaaaaggcccttagagggaatctagcccaacctacattccaccctgttacttatgtagaaactgaggcccagagagggaagatgacctgccccaagtggtgagcaagcaccaacctccagactcagcagagtgagggggtaaagcagttcctgtcccacatggccatcttctttcttccacccacaaactccaggctggaagtacttggcccccttcaggagcctggccaggcagggagagagtagctgcagccttcatcagaactcttcctcctcccaaggcattctcccagctctagcctctggactggaaagcacaagactggcccagtgccagcaagtccttaggctactgtaatgctgcctcaggacccatccctgcctggaggctcctctaggccctgtgagcacaaagaagaaagctgatttttgtcttttaatccatttcaggactctctccaggagggctcggggtgtgtcatttctatattcctccagctgggattggggggtgggctttgttgtgagaatggcctggagcaggcccaatgctgcttttgggggtcagcatccagtgtgagatactgtgtatataaactatatataatgtatataaactgggatgtaagtttgtgtaaattaatgttttattctttgcaaataaaacgctttccccgtcaaaaaaaaaaaaaaaaaaaaaaaaaaaaaaaaaaaaa

SEQ ID NO: 7 - DNA, 972 bp, Homo sapiens
  ref <308>: DATABASE ACCESSION NUMBER: NCBI/U11821.1
  ref <309>: DATABASE ENTRY DATE: 1995-04-14
  ref <313>: RELEVANT RESIDUES: (1)..(972)
  ref <308>: DATABASE ACCESSION NUMBER: NCBI/BC017502.1
  ref <309>: DATABASE ENTRY DATE: 2001-11-21
  ref <313>: RELEVANT RESIDUES: (1)..(972)
  ref <308>: DATABASE ACCESSION NUMBER: NCBI/NM_000639.1
  ref <309>: DATABASE ENTRY DATE: 2000-10-31
  ref <313>: RELEVANT RESIDUES: (1)..(972)
  ref <308>: DATABASE ACCESSION NUMBER: NCBI/U08137.1
  ref <309>: DATABASE ENTRY DATE: 1995-01-17
  ref <313>: RELEVANT RESIDUES: (1)..(972)
tctagactcaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagatgttctaga

SEQ ID NO: 8 - DNA, 3362 bp, Homo sapiens
  ref <308>: DATABASE ACCESSION NUMBER: NCBI/D90224.1
  ref <309>: DATABASE ENTRY DATE: 1999-02-07
  ref <313>: RELEVANT RESIDUES: (1)..(3362)
  ref <308>: DATABASE ACCESSION NUMBER: NCBI/NM_003326.2
  ref <309>: DATABASE ENTRY DATE: 2002-10-07
  ref <313>: RELEVANT RESIDUES: (1)..(3362)
  ref <308>: DATABASE ACCESSION NUMBER: NCBI/AL022310.1
  ref <309>: DATABASE ENTRY DATE: 1999-11-23
  ref <313>: RELEVANT RESIDUES: (1)..(3362)
  ref <308>: DATABASE ACCESSION NUMBER: NCBI/X79929.1
  ref <309>: DATABASE ENTRY DATE: 1994-08-22
  ref <313>: RELEVANT RESIDUES: (1)..(3362)
  ref <308>: DATABASE ACCESSION NUMBER: NCBI/AB042987.1
  ref <309>: DATABASE ENTRY DATE: 2001-06-02
  ref <313>: RELEVANT RESIDUES: (1)..(3362)
ccatatcttcatcttccctctacccagattgtgaagatggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttgaggggctgatggcaatatctaaaaccaggcaccagcatgaacaccaagctgggggtggacagggcatggattcttcattgcaagtgaaggagcctcccagctcagccacgtgggatgtgacaagaagcagatcctggccctcccgcccccacccctcagggatatttaaaacttattttatataccagttaatcttatttatccttatattttctaaattgcctagccgtcacaccccaagattgccttgagcctactaggcacctttgtgagaaagaaaaaatagatgcctcttcttcaagatgcattgtttctattggtcaggcaattgtcataataaacttatgtcattgaaaacggtacctgactaccatttgctggaaatttgacatgtgtgtggcattatcaaaatgaagaggagcaaggagtgaaggagtggggttatgaatctgccaaaggtggtatgaaccaacccctggaagccaaagcggcctctccaaggttaaattgattgcagtttgcatattgcctaaatttaaactttctcatttggtgggggttcaaaagaagaatcagcttgtgaaaaatcaggacttgaagagagccgtctaagaaataccacgtgctttttttctttaccattttgctttcccagcctccaaacatagttaatagaaatttcccttcaaagaactgtctggggatgtgatgctttgaaaaatctaatcagtgacttaagagagattttcttgtatacagggagagtgagataacttattgtgaagggttagctttactgtacaggatagcagggaactggacatctcagggtaaaagtcagtacggattttaatagcctggggaggaaaacacattctttgccacagacaggcaaagcaacacatgctcatcctcctgcctatgctgagatacgcactcagctccatgtcttgtacacacagaaacattgctggtttcaagaaatgaggtgatcctattatcaaattcaatctgatgtcaaatagcactaagaagttattgtgccttatgaaaaataatgatctctgtctagaaataccatagaccatatatagtctcacattgataattgaaactagaagggtctatatcagcctatgccagggcttcaatggaatagtatccccttatgtttagttgaaatgtccccttaacttgatataatgtgttatgcttatggcgctgtgacaatctgatttttcatgtcaacttccagatgatttgtaacttctctgtgccaaaccttttataaacataaatttttgagatatgtattttaaaattgtagcacatgtttccctgacattttcaatagaggatacaacatcacagaatctttctggatgattctgtgttatcaaggaattgtactgtgctacaattatctctagaatctccagaaaggtggagggctgttcgcccttacactaaatggtctcagttggatttttttttcctgttttctatttcctcttaagtacaccttcaactatattcccatccctctattttaatctgttatgaaggaaggtaaataaaaatgctaaatagaagaaattgtaggtaaggtaagaggaatcaagttctgagtggctgccaaggcactcacagaatcataatcatggctaaatatttatggagggcctactgtggaccaggcactggctaaatacttacatttacaagaatcattctgagacagatattcaatgatatctggcttcactactcagaagattgtgtgtgtgtttgtgtgtgtgtgtgtgtgtgtatttcactttttgttattgaccatgttctgcaaaattgcagttactcagtgagtgatatccgaaaaagtaaacgtttatgactataggtaatatttaagaaaatgcatggttcatttttaagtttggaatttttatctatatttctcacagatgtgcagtgcacatgcaggcctaagtatatgttgtgtgtgtttgtctttgacgtcatggtcccctctcttaggtgctcactcgctttgggtgcacctggcctgctcttcccatgttggcctctgcaaccacacagggatatttctgctatgcaccagcctcactccaccttccttccatcaaaaatatgtgtgtgtgtctcagtccctgtaagtcatgtccttcacagggagaattaacccttcgatatacatggcagagttttgtgggaaaagaattgaatgaaaagtcaggagatcagaattttaaatttgacttagccactaactagccatgtaaccttgggaaagtcatttcccatttctgggtcttgcttttctttctgttaaatgagaggaatgttaaatatctaacagtttagaatcttatgcttacagtgttatctgtgaatgcacatattaaatgtctatgttcttgttgctatgagtcaaggagtgtacacttctcctttactatgttgaatgtatttttttctggacaagcttacatcttcctcagccatctttgtgagtccttcaagagcagttatcaattgttagttagatattttctatttagagaatgcttaagggattccaatcccgatccaaatcataatttgttcttaagtatactgggcaggtcccctattttaagtcataattttgtatttagtgctttcctggctctcagagagtattaatattgatattaataatatagttaatagtaatattgctatttacatggaaacaaataaaagatctcagaattc

SEQ ID NO: 9 - DNA, 534 bp, Homo sapiens
  ref <308>: DATABASE ACCESSION NUMBER: NCBI/NM_005092.1
  ref <309>: DATABASE ENTRY DATE: 2002-10-07
  ref <313>: RELEVANT RESIDUES: (1)..(534)
  ref <308>: DATABASE ACCESSION NUMBER: NCBI/AF125303.1
  ref <309>: DATABASE ENTRY DATE: 1999-07-02
  ref <313>: RELEVANT RESIDUES: (1)..(534)
  ref <308>: DATABASE ACCESSION NUMBER: NCBI/AF117713.1
  ref <309>: DATABASE ENTRY DATE: 1999-08-09
  ref <313>: RELEVANT RESIDUES: (1)..(534)
  ref <308>: DATABASE ACCESSION NUMBER: NCBI/AL031599.1
  ref <309>: DATABASE ENTRY DATE: 1999-11-23
  ref <313>: RELEVANT RESIDUES: (1)..(534)
atgtgtttgagccacttggaaaatatgcctttaagccattcaagaactcaaggagctcagagatcatcctggaagctgtggctcttttgctcaatagttatgttgctatttctttgctccttcagttggctaatctttatttttctccaattagagactgctaaggagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctag

SEQ ID NO: 10 - DNA, 1906 bp, Homo sapiens
  ref <308>: DATABASE ACCESSION NUMBER: NCBI/NM_001244.1
  ref <309>: DATABASE ENTRY DATE: 2000-10-31
  ref <313>: RELEVANT RESIDUES: (1)..(1906)
  ref <308>: DATABASE ACCESSION NUMBER: NCBI/L09753.1
  ref <309>: DATABASE ENTRY DATE: 1995-08-23
  ref <313>: RELEVANT RESIDUES: (1)..(1906)
  ref <308>: DATABASE ACCESSION NUMBER: NCBI/AL133412.7
  ref <309>: DATABASE ENTRY DATE: 2002-10-04
  ref <313>: RELEVANT RESIDUES: (1)..(1906)
  ref <308>: DATABASE ACCESSION NUMBER: NCBI/AF006384.1
  ref <309>: DATABASE ENTRY DATE: 1998-01-28
  ref <313>: RELEVANT RESIDUES: (1)..(1906)
ccaagtcacatgattcaggattcagggggagaatccttcttggaacagagatgggcccagaactgaatcagatgaagagagataaggtgtgatgtggggaagactatataaagaatggacccagggctgcagcaagcactcaacggaatggcccctcctggagacacagccatgcatgtgccggcgggctccgtggccagccacctggggaccacgagccgcagctatttctatttgaccacagccactctggctctgtgccttgtcttcacggtggccactattatggtgttggtcgttcagaggacggactccattcccaactcacctgacaacgtccccctcaaaggaggaaattgctcagaagacctcttatgtatcctgaaaagagctccattcaagaagtcatgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagactgaacagtttctcttggccttcaggaagaaagcgcctctctaccatacagtatttcatccctccaaacacttgggcaaaaagaaaactttagaccaagacaaactacacagggtattaaatagtatacttctccttctgtctcttggaaagatacagctccagggttaaaaagagagtttttagtgaagtatctttcagatagcaggcagggaagcaatgtagtgtggtgggcagagccccacacagaatcagaagggatgaatggatgtcccagcccaaccactaattcactgtatggtcttgatctatttcttctgttttgagagcctccagttaaaatggggcttcagtaccagagcagctagcaactctgccctaatgggaaatgaaggggagctgggtgtgagtgtttacactgtgcccttcacgggatacttcttttatctgcagatggcctaatgcttagttgtccaagtcgcgatcaaggactctctcacacaggaaacttccctatactggcagatacacttgtgactgaaccatgcccagtttatgcctgtctgactgtcactctggcactaggaggctgatcttgtactccatatgaccccacccctaggaacccccagggaaaaccaggctcggacagccccctgttcctgagatggaaagcacaaatttaatacaccaccacaatggaaaacaagttcaaagacttttacttacagatcctggacagaaagggcataatgagtctgaagggcagtcctccttctccaggttacatgaggcaggaataagaagtcagacagagacagcaagacagttaacaacgtaggtaaagaaatagggtgtggtcactctcaattcactggcaaatgcctgaatggtctgtctgaaggaagcaacagagaagtggggaatccagtctgctaggcaggaaagatgcctctaagttcttgtctctggccagaggtgtggtatagaaccagaaacccatatcaagggtgactaagcccggcttccggtatgagaaattaaacttgtatacaaaatggttgccaaggcaacataaaattataagaattc

SEQ ID NO: 11 - DNA, 2785 bp, Homo sapiens
  misc_feature (49)..(49): n = a, t, c or g
  ref <308>: DATABASE ACCESSION NUMBER: NCBI/ AF039390
  ref <309>: DATABASE ENTRY DATE: 1999-01-22
  ref <313>: RELEVANT RESIDUES: (1)..(2785)
  ref <308>: DATABASE ACCESSION NUMBER: NCBI/ AL390240
  ref <309>: DATABASE ENTRY DATE: 2002-01-10
  ref <313>: RELEVANT RESIDUES: (1)..(2785)
  ref <308>: DATABASE ACCESSION NUMBER: NCBI/ NM_005118.2
  ref <309>: DATABASE ENTRY DATE: 2002-10-07
  ref <313>: RELEVANT RESIDUES: (1)..(2785)
agtgcagtatctcatggaggtgtttggatgtctcttcctgtggggggtnccaaagcccatgtctcttggcattttctttcagattctatcagccctctctctttctctcctgtctctctctttcattcatacactgagtcattcagagatggcttctctccaactcggagctgcaagtaattctggatctggtcacacacacaaagtccccagagttgccaatttatctagttcatctgtgcctgttcaagatgatgtaactaaacatttaccttcagggaggtgtttccaaagaattttcatcgatatatagaaatcaagagaaaatccatactatcaccaaatcaagagaaattccatactatcaccagttggccaactttccaagtctagtgcagaaatccaaggcacctcacacctagagttcctatacctctgagactccagaggaaagaacaagacagtgcagaaggatatgttagaacccactgaaaacctagaaggttaaaaaggaagcataccctcctgacctataagaaaattttcagtctgcagggggatatccttgtggcccaagacattggtgttatcatttgactaagaggaaattatttgtggtgagctctgagtgaggattaggaccagggagatgccaagtttctatcacttacctcatgcctgtaagacaagtgttttgttccaattgatgaatggggataaaacagttcagccaatcacttatggggcaaagaatgggaatttgaagggtctggtgcctggccttgtcatacgtaaacaagagaggcatcgatgagttttatctgagtcatttgggaaaggataattcttgcagcaagccattttcctaaacacagaagaatagggggattccttaaccttcattgttctccaggatcataggtctcaggtaaaattaaaaattttcaggtcagaccactcagtctcagaaaggcaaagtaatttgccccaggtcactagtccaagatgttattctctttgaacaaatgtgtatgtccagtcacatattcttcattcattcctccccaaagcagtttttagctgttaggtatattcgatcactttagtctattttgaaaatgatatgagacgctttttaagcaaagtctacagtttcccaatgagaaaattaatcctctttcttgtctttccagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactataggaggagagcaaatatcattatatgaaagtcctctgccaccgagttcctaattttctttgttcaaatgtaattataaccaggggttttcttggggccgggagtagggggcattccacagggacaacggtttagctatgaaatttggggccaaaatttcacacttcatgtgccttactgatgagagtactaactggaaaaaggctgaagagagcaaatatattattaagatgggttggaggattggcgagtttctaaatattaagacactgatcactaaatgaatggatgatctactcgggtcaggattgaaagagaaatatttcaacacctcctgctatacaatggtcaccagtggtccagttattgttcaatttgatcataaatttgcttcaattcaggagctttgaaggaagtccaaggaaagctctagaaaacagtataaactttcagaggcaaaatccttcaccaatttttccacatactttcatgccttgcctaaaaaaaatgaaaagagagttggtatgtctcatgaatgttcacacagaaggagttggttttcatgtcatctacagcatatgagaaaagctacctttcttttgattatgtacacagatatctaaataaggaagtatgagtttcacatgtatatcaaaaatacaacagttgcttgtattcagtagagttttcttgcccacctattttgtgctgggttctaccttaacccagaagacactatgaaaaacaagacagactccactcaaaatttatatgaacaccactagatacttcctgatcaaacatcagtcaacatactctaaagaataactccaagtcttggccaggcgcagtggctcacacctgtaatcccaacactttgggaggccaaggtgggtggatcatctaaggccgggagttcaagaccagcctgaccaacgtggagaaaccccatctctactaaaaatacaaaattagccgggcgtggtagcgcatggctgtaatcctggctactcaggaggccgaggcagaagaattgcttgaactggggaggcagaggttgcggtgagcccagatcgcgccattgcactccagcctgggtaacaagagcaaaactctgtccaaaaaaaaaaaaaaaaaaaa

SEQ ID NO: 12 - DNA, 1169 bp, Homo sapiens
  ref <308>: DATABASE ACCESSION NUMBER: NCBI/AF036581
  ref <309>: DATABASE ENTRY DATE: 1998-01-28
  ref <313>: RELEVANT RESIDUES: (1)..(1169)
  ref <308>: DATABASE ACCESSION NUMBER: NCBI/AF064090
  ref <309>: DATABASE ENTRY DATE: 1998-07-02
  ref <313>: RELEVANT RESIDUES: (1)..(1169)
  ref <308>: DATABASE ACCESSION NUMBER: NCBI/BC018058
  ref <309>: DATABASE ENTRY DATE: 2001-12-06
  ref <313>: RELEVANT RESIDUES: (1)..(1169)
  ref <308>: DATABASE ACCESSION NUMBER: NCBI/AY028261.1
  ref <309>: DATABASE ENTRY DATE: 2002-01-16
  ref <313>: RELEVANT RESIDUES: (1)..(1169)
gaggttgaaggacccaggcgtgtcagccctgctccagagaccttgggcatggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggctgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggaggaggtggtcgtccgtgtgctggatgaacgcctggttcgactgcgtgatggtacccggtcttacttcggggctttcatggtgtgaaggaaggagcgtggtgcattggacatgggtctgacacgtggagaactcagagggtgcctcaggggaaagaaaactcacgaagcagaggctgggcgtggtggctctcgcctgtaatcccagcactttgggaggccaaggcaggcggatcacctgaggtcaggagttcgagaccagcctggctaacatggcaaaaccccatctctactaaaaatacaaaaattagccggacgtggtggtgcctgcctgtaatccagctactcaggaggctgaggcaggataattttgcttaaacccgggaggcggaggttgcagtgagccgagatcacaccactgcactccaacctgggaaacgcagtgagactgtgcctcaaaaaaaaaaaaaaaaaaaaaaaaaaaaa

SEQ ID NO: 13 - DNA, 1619 bp, Homo sapiens
  ref <308>: DATABASE ACCESSION NUMBER: NCBI/NM_003811.1
  ref <309>: DATABASE ENTRY DATE: 2001-08-09
  ref <313>: RELEVANT RESIDUES: (1)..(1619)
  ref <308>: DATABASE ACCESSION NUMBER: NCBI/U03398
  ref <309>: DATABASE ENTRY DATE: 1994-11-27
  ref <313>: RELEVANT RESIDUES: (1)..(1619)
gtcatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataacgcccagcctgggtgcagcccacctggacagagtccgaatcctactccatccttcatggagacccctggtgctgggtccctgctgctttctctacctcaaggggcttggcaggggtccctgctgctgacctccccttgaggaccctcctcacccactccttccccaagttggaccttgatatttattctgagcctgagctcagataatatattatatatattatatatatatatatatttctatttaaagaggatcctgagtttgtgaatggacttttttagaggagttgttttggggggggggtcttcgacattgccgaggctggtcttgaactcctggacttagacgatcctcctgcctcagcctcccaagcaactgggattcatcctttctattaattcattgtacttatttgcctatttgtgtgtattgagcatctgtaatgtgccagcattgtgcccaggctagggggctatagaaacatctagaaatagactgaaagaaaatctgagttatggtaatacgtgaggaatttaaagactcatccccagcctccacctcctgtgtgatacttgggggctagcttttttctttctttcttttttttgagatggtcttgttctgtcaaccaggctagaatgcagcggtgcaatcatgagtcaatgcagcctccagcctcgacctcccgaggctcaggtgatcctcccatctcagcctctcgagtagctgggaccacagttgtgtgccaccacacttggctaactttttaatttttttgcggagacggtattgctatgttgccaaggttgtttacatgccagtacaatttataataaacactcatttttcc

SEQ ID NO: 14 - DNA, 926 bp, Homo sapiens
  ref <308>: DATABASE ACCESSION NUMBER: NCBI/L08096.1
  ref <309>: DATABASE ENTRY DATE: 1993-07-26
  ref <313>: RELEVANT RESIDUES: (1)..(926)
  ref <308>: DATABASE ACCESSION NUMBER: NCBI/NM_001252.1
  ref <309>: DATABASE ENTRY DATE: 2000-10-31
  ref <313>: RELEVANT RESIDUES: (1)..(926)
  ref <308>: DATABASE ACCESSION NUMBER: NCBI/S69339.1
  ref <309>: DATABASE ENTRY DATE: 1994-09-23
  ref <313>: RELEVANT RESIDUES: (1)..(926)
  ref <308>: DATABASE ACCESSION NUMBER: NCBI/BC000725.1
  ref <309>: DATABASE ENTRY DATE: 2001-07-12
  ref <313>: RELEVANT RESIDUES: (1)..(926)
ccagagaggggcaggcttgtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgtctcccagcgcctgacgcccctggcccgaggggacacactctgcaccaacctcactgggacacttttgccttcccgaaacactgatgagaccttctttggagtgcagtgggtgcgcccctgaccactgctgctgattagggttttttaaattttattttattttatttaagttcaagagaaaaagtgtacacacaggggccacccggggttggggtgggagtgtggtggggggtagtttgtggcaggacaagagaaggcattgagctttttctttcattttcctattaaaaaatacaaaaatcaaaacaaaaaaaa

SEQ ID NO: 15 - DNA, 894 bp, Homo sapiens
  ref <308>: DATABASE ACCESSION NUMBER: NCBI/U89922.1
  ref <309>: DATABASE ENTRY DATE: 1997-09-29
  ref <313>: RELEVANT RESIDUES: (1)..(894)
  ref <308>: DATABASE ACCESSION NUMBER: NCBI/NM_002341.1
  ref <309>: DATABASE ENTRY DATE: 2002-08-27
  ref <313>: RELEVANT RESIDUES: (1)..(894)
  ref <308>: DATABASE ACCESSION NUMBER: NCBI/L11015.1
  ref <309>: DATABASE ENTRY DATE: 1993-06-12
  ref <313>: RELEVANT RESIDUES: (1)..(894)
  ref <308>: DATABASE ACCESSION NUMBER: NCBI/NM_009588.1
  ref <309>: DATABASE ENTRY DATE: 2002-08-27
  ref <313>: RELEVANT RESIDUES: (1)..(894)
cagtctcaatgggggcactggggctggagggcaggggtgggaggctccaggggaggggttccctcctgctagctgtggcaggagccacttctctggtgaccttgttgctggcggtgcctatcactgtcctggctgtgctggccttagtgccccaggatcagggaggactggtaacggagacggccgaccccggggcacaggcccagcaaggactggggtttcagaagctgccagaggaggagccagaaacagatctcagccccgggctcccagctgcccacctcataggcgctccgctgaaggggcaggggctaggctgggagacgacgaaggaacaggcgtttctgacgagcgggacgcagttctcggacgccgaggggctggcgctcccgcaggacggcctctattacctctactgtctcgtcggctaccggggccgggcgccccctggcggcggggacccccagggccgctcggtcacgctgcgcagctctctgtaccgggcggggggcgcctacgggccgggcactcccgagctgctgctcgagggcgccgagacggtgactccagtgctggacccggccaggagacaagggtacgggcctctctggtacacgagcgtggggttcggcggcctggtgcagctccggaggggcgagagggtgtacgtcaacatcagtcaccccgatatggtggacttcgcgagagggaagaccttctttggggccgtgatggtggggtgagggaatatgagtgcgtggtgcgagtgcgtgaatattgggggcccggacgcccaggaccccatggcagtgggaaaaatgtaggagactgtttggaaattgattttgaacctgatgaaaataaagaatggaaagcttcagtgctgccgataaa

SEQ ID NO: 16 - DNA, 1306 bp, Homo sapiens
  ref <308>: DATABASE ACCESSION NUMBER: NCBI/AF030099.1
  ref <309>: DATABASE ENTRY DATE: 1997-12-20
  ref <313>: RELEVANT RESIDUES: (1)..(1306)
  ref <308>: DATABASE ACCESSION NUMBER: NCBI/NM_003809.2
  ref <309>: DATABASE ENTRY DATE: 2002-10-07
  ref <313>: RELEVANT RESIDUES: (1)..(1306)
  ref <308>: DATABASE ACCESSION NUMBER: NCBI/AF055872.1
  ref <309>: DATABASE ENTRY DATE: 1998-05-04
  ref <313>: RELEVANT RESIDUES: (1)..(1306)
  ref <308>: DATABASE ACCESSION NUMBER: NCBI/BC019047.1
  ref <309>: DATABASE ENTRY DATE: 2001-12-11
  ref <313>: RELEVANT RESIDUES: (1)..(1306)
  ref <308>: DATABASE ACCESSION NUMBER: NCBI/AF030100.1
  ref <309>: DATABASE ENTRY DATE: 1997-12-20
  ref <313>: RELEVANT RESIDUES: (1)..(1306)
cacagccccccgcccccatggccgcccgtcggagccagaggcggagggggcgccggggggagccgggcaccgccctgctggtcccgctcgcgctgggcctgggcctggcgctggcctgcctcggcctcctgctggccgtggtcagtttggggagccgggcatcgctgtccgcccaggagcctgcccaggaggagctggtggcagaggaggaccaggacccgtcggaactgaatccccagacagaagaaagccaggatcctgcgcctttcctgaaccgactagttcggcctcgcagaagtgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggccagttccctcgggccccagctccgcctctgccaggtgtctgggctgttggccctgcggccagggtcctccctgcggatccgcaccctcccctgggcccatctcaaggctgcccccttcctcacctacttcggactcttccaggttcactgaggggccctggtctccccacagtcgtcccaggctgccggctcccctcgacagctctctgggcacccggtcccctctgccccaccctcagccgctctttgctccagacctgcccctccctctagaggctgcctgggcctgttcacgtgttttccatcccacataaatacagtattcccactcttatcttacaactcccccaccgcccactctccacctcactagctccccaatccctgaccctttgaggcccccagtgatctcgactcccccctggccacagacccccagggcattgtgttcactgtactctgtgggcaaggatgggtccagaagaccccacttcaggcactaagaggggctggacctggcggcaggaagccaaagagactgggcctaggccaggagttcccaaatgtgaggggcgagaaacaagacaagctcctcccttgagaattccctgtggatttttaaaacagatattatttttattattattgtgacaaaatgttgataaatggatattaaatagaataagtcag

SEQ ID NO: 17 - DNA, 1348 bp, Homo sapiens
  ref <308>: DATABASE ACCESSION NUMBER: NCBI/NM_003808.1
  ref <309>: DATABASE ENTRY DATE: 2002-08-27
  ref <313>: RELEVANT RESIDUES: (1)..(1348)
  ref <308>: DATABASE ACCESSION NUMBER: NCBI/AF046888.1
  ref <309>: DATABASE ENTRY DATE: 1998-09-25
  ref <313>: RELEVANT RESIDUES: (1)..(1348)
  ref <308>: DATABASE ACCESSION NUMBER: NCBI/AF114011.1
  ref <309>: DATABASE ENTRY DATE: 2000-09-19
  ref <313>: RELEVANT RESIDUES: (1)..(1348)
  ref <308>: DATABASE ACCESSION NUMBER: NCBI/AF184972.1
  ref <309>: DATABASE ENTRY DATE: 2000-01-13
  ref <313>: RELEVANT RESIDUES: (1)..(1348)
  ref <308>: DATABASE ACCESSION NUMBER: NCBI/AF114013.1
  ref <309>: DATABASE ENTRY DATE: 2000-03-25
  ref <313>: RELEVANT RESIDUES: (1)..(1348)
ggtacgaggcttcctagagggactggaacctaattctcctgaggctgagggagggtggagggtctcaaggcaacgctggccccacgacggagtgccaggagcactaacagtacccttagcttgctttcctcctccctcctttttattttcaagttcctttttatttctccttgcgtaacaaccttcttcccttctgcaccactgcccgtacccttacccgccccgccacctccttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgattgtgttataaaaagtggctcccagcttggaagaccagggtgggtacatactggagacagccaagagctgagtatataaaggagagggaatgtgcaggaacagaggcatcttcctgggtttggctccccgttcctcacttttcccttttcattcccaccccctagactttgattttacggatatcttgcttctgttccccatggagctccgaattcttgcgtgtgtgtagatgaggggcgggggacgggcgccaggcattgttcagacctggtcggggcccactggaagcatccagaacagcaccaccatctta

SEQ ID NO: 18 - DNA, 1090 bp, Homo sapiens
  ref <308>: DATABASE ACCESSION NUMBER: NCBI/NM_006573.3
  ref <309>: DATABASE ENTRY DATE: 2002-10-07
  ref <313>: RELEVANT RESIDUES: (1)..(1090)
  ref <308>: DATABASE ACCESSION NUMBER: NCBI/AF134715.1
  ref <309>: DATABASE ENTRY DATE: 2000-03-28
  ref <313>: RELEVANT RESIDUES: (1)..(1090)
  ref <308>: DATABASE ACCESSION NUMBER: NCBI/AF186114.1
  ref <309>: DATABASE ENTRY DATE: 2000-01-13
  ref <313>: RELEVANT RESIDUES: (1)..(1090)
  ref <308>: DATABASE ACCESSION NUMBER: NCBI/AF132600.1
  ref <309>: DATABASE ENTRY DATE: 1999-03-21
  ref <313>: RELEVANT RESIDUES: (1)..(1090)
  ref <308>: DATABASE ACCESSION NUMBER: NCBI/AF116456.1
  ref <309>: DATABASE ENTRY DATE: 2002-01-22
  ref <313>: RELEVANT RESIDUES: (1)..(1090)
  ref <308>: DATABASE ACCESSION NUMBER: NCBI/AY129225.1
  ref <309>: DATABASE ENTRY DATE: 2002-09-17
  ref <313>: RELEVANT RESIDUES: (1)..(1090)
taactctcctgaggggtgagccaagccctgccatgtagtgcacgcaggacatcaacaaacacagataacaggaaatgatccattccctgtggtcacttattctaaaggccccaaccttcaaagttcaagtagtgatatggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtgacctacttacaccatgtctgtagctattttcctccctttctctgtacctctaagaagaaagaatctaactgaaaataccaaaaaaaaaaaaaaaaaa

SEQ ID NO: 19 - protein, 316 aa, Homo sapiens
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENAGLQDSTLESEDTLPDSCRRMKQAFQGAVQKELQHIVGPQRFSGAPAMMEGSWLDVAQRGKPEAQPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQDID

SEQ ID NO: 20 - protein, 24 aa, Homo sapiens
NAASIPSGSHKVTLSSWYHDRGWA

SEQ ID NO: 21 - protein, 10 aa, Homo sapiens
HETSGSVPTD

SEQ ID NO: 22 - protein, 7 aa, Homo sapiens
SIKIPSS

SEQ ID NO: 23 - protein, 9 aa, Homo sapiens
KNWSGNSEF

SEQ ID NO: 24 - protein, 34 aa, Homo sapiens
TRGRSNTLSSPNSKNEKALGRKINSWESSRSGHS

SEQ ID NO: 25 - protein, 10 aa, Homo sapiens
QEEIKENTKN

SEQ ID NO: 26 - protein, 6 aa, Homo sapiens
TSYPDP

SEQ ID NO: 27 - protein, 9 aa, Homo sapiens
SCWSKDAEY

SEQ ID NO: 28 - protein, 18 aa, Homo sapiens
EASSKTTSVLQWAEKGYY

SEQ ID NO: 29 - protein, 6 aa, Homo sapiens
NREASS

SEQ ID NO: 30 - protein, 6 aa, Homo sapiens
SPGRFE

SEQ ID NO: 31 - protein, 7 aa, Homo sapiens
HSSAKPC

SEQ ID NO: 32 - protein, 17 aa, Homo sapiens
NHQVEEQLEWLSQRANA

SEQ ID NO: 33 - protein, 5 aa, Homo sapiens
QGCPD

SEQ ID NO: 34 - protein, 7 aa, Homo sapiens
AISYQEK

SEQ ID NO: 35 - protein, 14 aa, Homo sapiens
PCPKDTPEGAELKP

SEQ ID NO: 36 - protein, 17 aa, Homo sapiens
DPSKQNSLLWRANTDRA

SEQ ID NO: 37 - protein, 10 aa, Homo sapiens
KAYSPKATSS

SEQ ID NO: 38 - protein, 7 aa, Homo sapiens
SSQYPFH

SEQ ID NO: 39 - protein, 8 aa, Homo sapiens
VYPGLQEP

SEQ ID NO: 40 - protein, 17 aa, Homo sapiens
ETRVTVPNVPIRFTKIF

SEQ ID NO: 41 - protein, 1 aa, Homo sapiens
D

SEQ ID NO: 42 - protein, 4 aa, Homo sapiens
YQEK

SEQ ID NO: 43 - protein, 24 aa, Homo sapiens
NATDIPSGSHKVSLSSWYHDRGWA

SEQ ID NO: 44 - protein, 10 aa, Homo sapiens
HETSGDLATE

SEQ ID NO: 45 - protein, 7 aa, Homo sapiens
SIKIPSS

SEQ ID NO: 46 - protein, 9 aa, Homo sapiens
KYWSGNSEF

SEQ ID NO: 47 - protein, 17 aa, Homo sapiens
NPQAEGQLQWLNRRANA

SEQ ID NO: 48 - protein, 5 aa, Homo sapiens
QGCPS

SEQ ID NO: 49 - protein, 7 aa, Homo sapiens
AVSYQTK

SEQ ID NO: 50 - protein, 14 aa, Homo sapiens
PCQRETPEGAEAKP

SEQ ID NO: 51 - DNA, 2201 bp, Homo sapiens
ggccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccccgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacctgccagtgggagatgttagactcatggtgtgttacacaatggtttttaaattttgtaatgaattcctagaattaaaccagattggagcaattacgggttgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatttgtcaagagtagttatataattgttgaacaggtgtttttccacaagtgccgcaaattgtacctttttttttttttcaaaatagaaaagttattagtggtttatcagcaaaaaagtccaattttaatttagtaaatgttattttatactgtacaataaaaacattgcctttgaatgttaattttttggtacaaaaataaatttatatgaaaaaaaaaaaaaaaaaaaaaaaaaaaaaaaa

SEQ ID NO: 52 - protein, 24 aa, Homo sapiens
NATDIPSGSHKVSLSSWYHDRGWG